(19) United States
(12) Patent Application Publication
(10) Pub. No.: US 2022/0143124 A1
(43) Pub. Date: May 12, 2022

(54) SYNERGISTIC HERBAL COMPOSITIONS FOR PREVENTION, TREATMENT OR CONTROL OF PAIN AND/OR CARTILAGE REGENERATION

(71) Applicant: LAILA NUTRACEUTICALS, Vijayawada (IN)

(72) Inventors: Ganga Raju GOKARAJU, Vijayawada (IN); Rama Raju GOKARAJU, Vijayawada (IN); Venkata Kanaka Ranga Raju GOKARAJU, Vijayawada (IN); Trimurtulu GOLAKOTI, Vijayawada (IN); Kiran BHUPATHIEAJU, Vijayawada (IN); Krishanu SENGUPTHA, Vijayawada (IN); Vankata Krishna Raju ALLURI, Vijayawada (IN); Venkateswarlu SOMEPALLI, Vijayawada (IN)

(21) Appl. No.: 17/600,793

(22) PCT Filed: Apr. 1, 2020
(86) PCT No.: PCT/IN2020/050318

§ 371 Date: Oct. 1, 2021

(30) Foreign Application Priority Data
Apr. 3, 2019 (IN) 201941013491

(51) Int. Cl. A61K 36/75; A61K 36/23; A61K 36/9066; A61P 19/02

(52) U.S. Cl. CPC A61K 36/75; A61P 19/02; A61K 36/9066; A61K 36/23

(57) ABSTRACT

Disclosed herein is Synergistic herbal composition(s) comprising combination of a first ingredient selected from the extract(s), fraction(s), phytochemical(s) and mixtures thereof derived from Murraya koenigii and a second ingredient selected from the extract(s), fraction(s), phytochemical(s) and mixtures thereof derived from Coriandrum sativum or Curcuma amada for improving cartilage growth/repair/regeneration, improving joint mobility, reducing cartilage breakdown, reducing joint pain, reducing stiffness, reducing inflammation and/or treating/alleviating symptoms associated with osteoarthritis. The invention further provides method of improving cartilage growth/repair/regeneration, improving joint mobility, reducing cartilage breakdown, reducing joint pain, reducing stiffness, reducing inflammation and/or treating/alleviating symptoms associated with osteoarthritis in a human or animal.

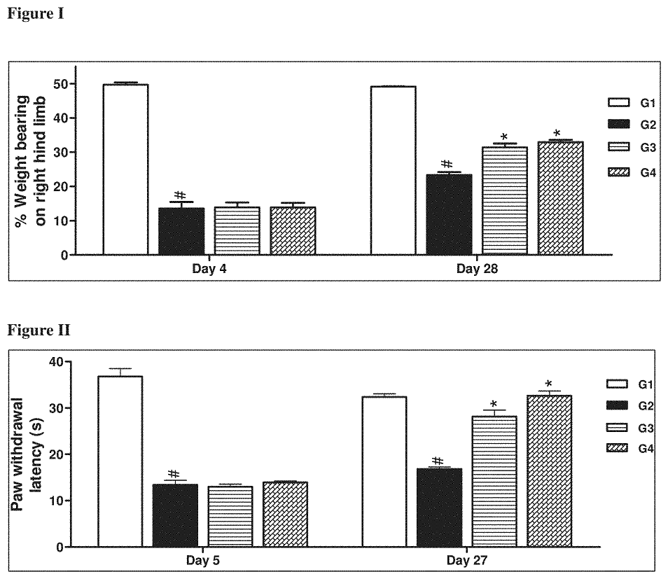

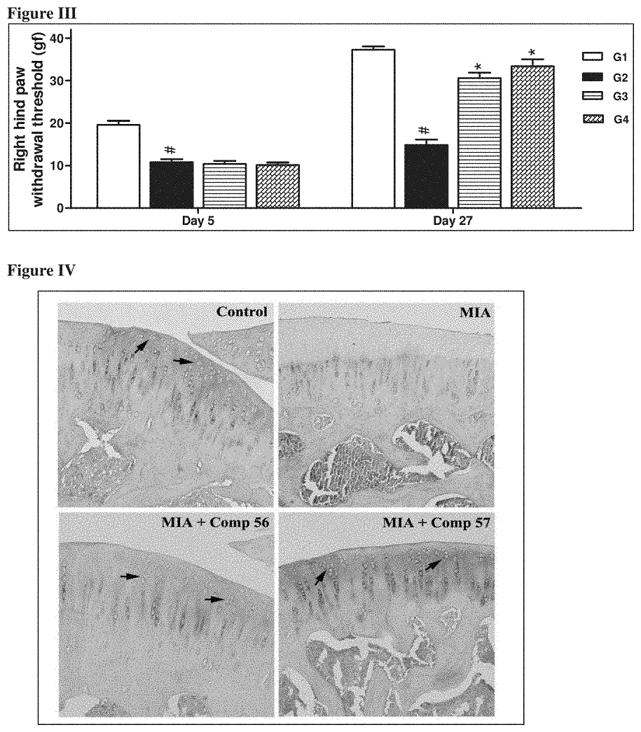

### TECHNICAL FIELD OF THE INVENTION

The invention relates to synergistic herbal compositions comprising combination of a first ingredient selected from the extract(s), fraction(s), phytochemical(s) and mixtures thereof derived from Murraya koenigii and a second ingredient selected from the extract(s), fraction(s), phytochemical(s) and mixtures thereof derived from Coriandrum sativum or Curcuma amada for obtaining at least one health benefit selected from improving cartilage build up/regeneration, reducing pain, reducing inflammation and treating/alleviating symptoms associated with osteoarthritis. The invention also relates to the method of improving cartilage build up/regeneration, reducing pain, reducing inflammation and/or treating/alleviating symptoms associated with osteoarthritis in a human.

### BACKGROUND OF THE INVENTION

Osteoarthritis (OA) is one of the most prevalent and chronic diseases affecting older adults. In a global estimate, about 10% population aged about 60 years or older have significant clinical presentations that are attributed to osteoarthritis (OA). OA is often progressive and disabling disease in developed and developing countries. The characteristic symptoms and signs of OA in the affected joints are heat, swelling, pain, stiffness and limited mobility.

Aging and inflammation are major contributing factors to the development and progression of OA. During physiological aging, low-grade inflammation occurs in chondrocytes of cartilage. The chondrocytes respond to inflammatory conditions and participate in the catabolic activities that ultimately lead to the degradation of cartilage extracellular matrix (ECM). Under inflammatory conditions, the chondrocytes actively produce pro-inflammatory cytokines, such as TNFα, IL-1β, IL-12, IL-15, and matrix-degrading enzymes. These catabolic factors generate metabolic imbalances in articular cartilage turnover (degradation and synthesis). In a majority, the breakdown and turnover of collagens in cartilage are mediated by the matrix metalloproteinase (MMPs). The MMPs are a group of extracellular proteinases which cleave the ECM as well as proteins on the cell surface and in pericellular regions. The synthesis and activity of MMPs in cartilage and other joint tissues is tightly controlled by a variety of pro-inflammatory cytokines, growth factors, and tissue inhibitors. MMPs are secreted as inactive proenzymes and are generally activated by other proteinases. MMP-13 is considered as the most important collagenase in the breakdown of cartilage and has a preference for type II collagen. Also, MMP3 is secreted from chondrocytes and synovial cells; it not only degrades ECM but also activates other serine proteases. Therefore, the development of the cartilage pathology in OA involves excessive damage to the collagen matrix, which is mediated primarily by the chondrocyte-generated pro-inflammatory cytokines such as TNFα, IL-1β, and the collagenases, MMP-13 and MMP-3. In concurrence with matrix degradation, the pro-inflammatory cytokines also downregulate the chondrogenic growth and thereby reduces the ECM synthesis. IL-1β has been shown to suppress ECM such as collagen type II and aggrecan synthesis via down-regulating a transcription factor, Sex-determining region Y-box 9 (SOX-9) in chondrocytes. SOX-9 is crucial for regulating the anabolic activities of chondrocytes and inducing chondrogenesis. The inflammatory cytokines not only affect the homeostatic functions of residential chondrocytes but also impact the chondrogenic differentiation of mesenchymal stem cells (MSCs).

Typically, the clinical presentation of OA is joint pain, involving central and peripheral mechanisms. The intensity of pain is directly correlated with the severity of joint degradation. OA pain is initiated from free nerve endings located in the synovium, periosteum, and tendons. One of the first effects of pro-inflammatory cytokines is the activation of phospholipase A2 (PLA2) which cleaves cellular membranes thereby liberating arachidonic acid (AA). AA is metabolized via three pathways forming different classes of eicosanoids acting in an autocrine/paracrine manner on target cells after extracellular release. Cyclooxygenases catalyze the formation of prostaglandins (PG) and Thromboxane (TX); Lipoxygenases (LO) form Leukotrienes (LT) and Lipoxins (LX); and Cytochrome P450 enzymes (CYP) produce epoxyeicosatrienoic acids (EET). Among prostaglandins and Leukotrienes PGE2 and LTB4, respectively, are the major eicosanoids. PGE2 is considered as a potent modulator of nociceptors for inflammatory pain perception. Therefore, the use of COX inhibitors, e.g., aspirin, ibuprofen like non-steroidal anti-inflammatory drugs (NSAIDs) is the first line choice for the alleviation of OA pain. However, the major NSAIDs' adverse drug reactions, such as gastric toxicity due to co-inhibition of COX-1 (constitutive isoform), limit its long term usage. Lipoxygenases, in particular the 5-LOX pathway derived LTB4 is a potent modulator of inflammatory reactions, induces pro-inflammatory cytokines TNFα, IL-1β, IL-6. LTB4 is also chemotactic for leukocytes, and it plays an important role in the development of gastrointestinal ulcers through its contribution to the inflammatory process. Therefore, it is plausible that the use of a COX/5-LOX dual inhibitor can provide more efficient relief from inflammatory pain (such as OA) with a better safety profile (in particular gastrointestinal) than the use of a COX alone inhibitors such as NSAIDs.

JP2009120512A discloses a dipeptide having a structure of Pro-Hyp, as a joint cartilage regeneration promoter, effective for prophylaxis or therapy of various symptoms such as osteoporosis and osteoarthritis as an active ingredient.

EP2319521B1 discloses a pharmaceutical composition comprises extracts of Chaenomelis Fructus, Achyranthis Radix, Acanthopanax, Cinnamomi Cortex, Gentianae Radix, Clematidis Radix, Angelicae Radix, Cnidii Rhizome, Gastrodiae Rhizoma, Safflower, Phlomidis Radix, and Ledebouriellae Radix, wherein the extracts contain acanthoside D as active ingredient, for use in pain suppression or edema suppression in osteoarthritis.

US20070071840A1 discloses a composition containing extract of Centella and a pharmaceutically acceptable carrier for the treatment of cartilage disorders in mammals.

The Indian patent application 201741016259 discloses a synergistic herbal formulation containing Aerva lanata, Boerhavia diffusa, and Murraya koenigii for anti-inflammatory and anti-oxidant activity.

US2016220627A1 discloses carbon dioxide extract of Curcuma amada for Cell proliferation disorder, inflammation, fever, infection, hyperlipidemia, hypercholesterolemia, hypertriglyceridemia, hyperglycemia, platelet hyper-aggregation, aged and/or sun-damaged skin, immune disorder, or neurodegenerative condition.

KR20110057049A discloses a composition contains Lepisorus thunbergianus, Coriandrum sativum, mistletoe Epimedii Herba, Cortex eucommiae, Juniperus rigida, Angelica keiskei, Glycyrrhiza, Zingiberis Rhizoma, and honey for prostatic hypertrophy, kidney, bladder, and urethra and to ensure detoxification and anti-inflammation.

As is evident from the above, there is a continuous need in the art to provide alternative and effective treatments comprising highly effective herbal compositions for the treatment of Osteoarthritis, joint pain and cartilage regeneration.

From a cursory review of the prior arts, there appears no knowledge relating to the compositions comprising the first active ingredient derived from Murraya koenigii and a second active ingredient derived from Coriandrum sativum or Curcuma amada for obtaining at least one health benefit selected from improving cartilage growth/repair/regeneration, reducing cartilage breakdown, reducing pain, reducing stiffness, reducing inflammation, improving joint mobility and treating/alleviating symptoms associated with osteoarthritis.

### OBJECT OF THE INVENTION

Therefore, the object of the present invention is to provide synergistic and safe herbal compositions comprising combination of a first ingredient selected from the extract(s), fraction(s), phytochemical(s) and mixtures thereof derived from Murraya koenigii and a second ingredient selected from the extract(s), fraction(s), phytochemical(s) and mixtures thereof derived from Coriandrum sativum or Curcuma amada for obtaining at least one health benefit selected from improving cartilage growth/repair/regeneration, reducing cartilage breakdown, reducing pain, reducing stiffness, reducing inflammation, improving joint mobility and treating/alleviating symptoms associated with osteoarthritis.

Another objective of the invention is to provide a method of obtaining at least one health benefit selected from improving cartilage growth/repair/regeneration, reducing cartilage breakdown, reducing pain, reducing stiffness, reducing inflammation, improving joint mobility and treating/alleviating symptoms associated with osteoarthritis in a human, wherein the method comprises supplementing said humans with an effective dose of synergistic composition comprising combination of a first ingredient selected from the extract(s), fraction(s), phytochemical(s) and mixtures thereof derived from Murraya koenigii and a second ingredient selected from the extract(s), fraction(s), phytochemical(s) and mixtures thereof derived from Coriandrum sativum or Curcuma amada.

Yet another objective of the invention is to provide use of the synergistic herbal compositions of the present inventions for obtaining at least one health benefit selected from improving cartilage growth/repair/regeneration, reducing cartilage breakdown, reducing pain, reducing stiffness, reducing inflammation, improving joint mobility and treating/alleviating symptoms associated with osteoarthritis.

### SUMMARY OF THE INVENTION

The present invention provides synergistic herbal compositions comprising combination of a first ingredient selected from the extract(s), fraction(s), phytochemical(s) and mixtures thereof derived from Murraya koenigii and a second ingredient selected from the extract(s), fraction(s), phytochemical(s) and mixtures thereof derived from Coriandrum sativum or Curcuma amada for obtaining at least one health benefit selected from improving cartilage growth/repair/regeneration, reducing cartilage breakdown, reducing pain, reducing stiffness, reducing inflammation, improving joint mobility and treating/alleviating symptoms associated with osteoarthritis in a human.

Another aspect of the invention provides synergistic herbal compositions comprising combination of a first ingredient selected from the extract(s), fraction(s), phytochemical(s) and mixtures thereof derived from Murraya koenigii and a second ingredient selected from the extract(s), fraction(s), phytochemical(s) and mixtures thereof derived from Coriandrum sativum or Curcuma amada; and optionally containing at least one component selected from pharmaceutically or nutraceutically or dietically acceptable excipients, carriers and diluents.

Other aspect of the invention provides methods of obtaining at least one health benefit selected from improving cartilage growth/repair/regeneration, reducing cartilage breakdown, reducing pain, reducing stiffness, reducing inflammation, improving joint mobility and treating/alleviating symptoms associated with osteoarthritis in a human, wherein the method comprises supplementing human with an effective dose of a composition comprising combination of first ingredient selected from the extract(s), fraction(s), phytochemical(s) and mixtures thereof derived from Murraya koenigii and second ingredient selected from the extract(s), fraction(s), phytochemical(s) and mixtures thereof derived from Coriandrum sativum or Curcuma amada; and optionally containing at least one component selected from pharmaceutically or nutraceutically or dietically acceptable excipients, carriers and diluents.

Another aspect of the invention provides the use of synergistic herbal compositions comprising combination of first ingredient selected from the extract(s), fraction(s), phytochemical(s) and mixtures thereof derived from Murraya koenigii and second ingredient selected from the extract(s), fraction(s), phytochemical(s) and mixtures thereof derived from Coriandrum sativum or Curcuma amada; and optionally containing at least one component selected from pharmaceutically or nutraceutically or dietically acceptable excipients, carriers and diluents for obtaining at least one health benefit selected from improving cartilage growth/repair/regeneration, reducing cartilage breakdown, reducing pain, reducing stiffness, reducing inflammation, improving joint mobility and treating/alleviating symptoms associated with osteoarthritis.

### DESCRIPTION OF FIGURES

FIG. I: Each bar represents the mean±SE of percent body weight bearing on the right hind limb of the experimental rats. G1, Vehicle control animals received (intra-articular) sterile normal saline; G2, MIA induced; G3, and G4 are MIA induced animals supplemented with Composition-56 (250 mg/kg), and Composition-57 (250 mg/kg), respectively. n=6; #, p<0.05 vs. vehicle control (G1); *, p<0.05 vs. MIA (G2); Two way ANOVA followed by Bonferroni test.

FIG. II: Each bar represents the mean±SE of the latent period (in seconds) to withdraw the paw after giving heat stimulus. G1, Vehicle control animals received (intra-articular) sterile normal saline; G2, MIA induced; G3, and G4 are MIA induced animals supplemented with Composition-56 (250 mg/kg), and Composition-57 (250 mg/kg), respectively. n=6; #, p<0.05 vs. vehicle control (G1); *, p<0.05 vs. MIA (G2); Two way ANOVA followed by Bonferroni test.

FIG. III: Each bar represents the mean±SE of the pressure threshold (in gram-force, gf) to withdraw the right hind paw. G1, Vehicle control animals received (intra-articular) sterile normal saline; G2, MIA induced; G3 and G4 are MIA induced animals supplemented with Composition-56 (250 mg/kg) and Composition-57 (250 mg/kg) respectively. n=6; #, p<0.05 vs. vehicle control (G1); *, p<0.05 vs. MIA (G2); Two way ANOVA followed by Bonferroni test.

FIG. IV: The representative photomicrographs show histologic changes and modulation of extracellular matrix (ECM) components in the knee joint articular cartilage sections stained with Safranin O green and counter-stained with fast green. Magnification ×200; arrows indicate articular cellular structures, chondrocytes. The supplementation of the animals with Composition-56 (G3) and Composition-57 (G4) helped to improve the joint architecture.

### DETAILED DESCRIPTION OF THE INVENTION

The invention will now be described in detail in connection with certain preferred and optional embodiments, so that various aspects thereof may be more fully understood and appreciated.

Osteoarthritis (OA) is a very prevalent and chronic disease affecting older adults. The characteristic features and symptoms of OA in the affected joints are decrease in cartilage growth/repair/regeneration, reduced joint mobility, cartilage breakdown, joint pain, stiffness, and inflammation. To address the problem and to provide a safe herbal composition(s) for improving cartilage growth/repair/regeneration, improving joint mobility, reducing cartilage breakdown, reducing joint pain, reducing stiffness, reducing inflammation and/or treating/alleviating symptoms associated with osteoarthritis, the following cell based assays have been chosen.

(a) Recovery of SOX-9 for regeneration of cartilage.

(b) Inhibition of MMP-13 to stop cartilage degradation.

(c) Inhibition of PGE2 and inhibition of LTB 4 to relieve pain and inflammation.

SOX-9: SRY-related high-mobility-group box 9 (SOX-9) is a cartilage-specific transcription factor, which plays essential role in chondrocyte differentiation and cartilage formation. SOX-9 positively modulates the expressions of several cartilage-specific ECM components including aggrecan and collagens II, IX, and XI. Recently, it has been shown that SOX-9 regulates the chondrogenesis process from Mesenchymal stem cells (MSCs). In progressive OA, gradual degeneration of articular cartilage leads to increasing severity of joint pain and dysfunction. Hence, the current evidences suggest that a positive modulation of this chondrogenic master regulator (SOX-9) is a potential tool in improving cartilage regeneration/growth/repair in progressive OA.

Matrix metallopeptidase 13 (MMP-13): Matrix metalloproteinase (MMP) 13 is a major enzyme that triggers proteolytic degradation of cartilage. Inflammatory cytokines e.g. IL-1β and other pro-inflammatory modulators such as PGE2 are the major players, which escalates MMP-13 production. The expression of MMP-13 is usually restricted to connective tissue and its overexpression is identified as a critical step during the progression of OA. It not only targets type II collagen in cartilage for degradation, but also degrades other structural proteins such as proteoglycan, collagen types IV and IX, osteonectin and perlecan. Clinical investigations reveal that the patients with damaged articular cartilage have high MMP-13 expression. This suggests that increased MMP13 may be associated with cartilage degradation. MMP-13 along with other MMPs plays significant role in gradual cartilage degradation causing severity in progressive OA. Therefore, strategies for inhibition of MMP-13 have drawn significant attention for developing potential therapies for the management of progressive OA.

Prostaglandin E2 (PGE2): Prostaglandins (PGs) are lipid compounds called eicosanoids, which are derived from arachidonic acid released from lipid membranes following activation of phospholipase A2 enzyme. Cyclooxygenase-2 (COX-2) is the key enzyme in prostaglandin E2 (PGE2) synthesis during inflammation. In inflammatory joints or in OA, PGE2 level is remarkably elevated in the synovial fluid. PGE2 increases sensitivity of peripheral nociceptive primary afferent neurons and central nociceptive neurons, hence, contributes to the chronic disabling pain in arthritic joints. PGE2 also contributes synovial inflammation in OA by increasing local blood flow and potentiates the effects of bradykinin and interleukin (IL)-1β to induce vascular permeability. Hence, PGE2 inhibition is as an effective strategy, e.g. as in non-steroidal anti-inflammatory drugs (NSAIDs), to relieve pain and inflammation. PGE2 exerts its effects via a variety of E prostanoid (EP) receptors such as EP1, EP2, EP3 and EP4. In addition, PGE2 also increases the production of matrix degrading enzymes such as MMP-3, MMP-13 etc.

Leukotriene B4: Leukotriene B4 (LTB4) is a pro-inflammatory lipid mediator, also synthesized from arachidonic acid via activation of 5-lipoxygenase (5-LOX). LTB4 is one of the potent mediators of inflammation, causing increased activation, recruitment, migration and adhesion of immune cells. Studies have shown that the LTB4 level is higher in the joints of OA patients in comparison to the levels observed in healthy individuals. Increased synthesis of LTB4 plays a pathogenic role that contributes to the pain and inflammation of OA. LTB4 antagonism is one of the promising strategies to alleviate inflammation and pain in OA patients. Recently, it has been proposed that the drug candidates, which are capable of inhibiting both 5-lipoxygenase and cyclooxygenases, would possess analgesic and anti-inflammatory properties. Recent clinical evidences suggest that these dual inhibitors have better safety profile compared with current pain management strategies using the nonselective COX-1/COX-2 inhibitors such as aspirin, ibuprofen, and naproxen.

Hence, the inventors of the current application randomly screened a large number of plant extracts and fractions for their SOX-9 recovery, MMP-13 inhibition, PGE2 inhibition and LTB4 inhibition activities using in vitro cellular models and found that the extract(s) and fraction(s) derived from Murraya koenigii, Coriandrum sativum and Curcuma amada show potent dose dependent efficacy.

A brief summary on each of the plant material is provided herein below. Murraya koenigii (L) (Fam. Rutaceae), commonly known as curry-leaf tree is a native of India, Sri Lanka and other South Asian countries. Curry leaf is widely used in Indian cuisine for its aroma and flavor. Traditionally leaves of this plant are used externally for application on bruises, burns, eruption, and treatment of bites of poisonous animals. It is used internally to cure dysentery and Diabetes mellitus. Ayurvedic system of medicine suggests powdered dry curry leaf mixed with honey and betel nut juice as an anti-periodic. Various pharmacological activities have been reported on medicinal uses of curry leaves such as Vasodilation, Anti-diabetic, Hypocholesteremic, Anti-ulcer, Anti-Diarrheal, Phagocytic, Analgesic and Antinociceptive activity, radio and chemo protective, memory enhancing, wound healing etc. (H. K. Handral et al., Asian Journal of Pharmaceutical and Clinical Research, 2012, 5, 5-14; V. Jain et al., International Journal of Ayurvedic & Herbal Medicine, 2012, 2(4), 607-627). Mahanine is one of the major carbazole alkaloid compounds in Murraya koenigii and the curry leaf extracts used in the current invention are standardized to Mahanine by HPLC method of analysis. Standardization can be extended to other alkaloid compounds in the curry leaf such as Mahanimbine, isomahanine, mahanibidine, koenimbine, murrayanol.

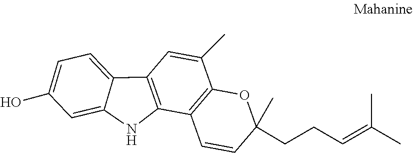 [SMILES: CC(C)=CCCC1(C)C=Cc2c(c(C)cc3c2[nH]c2cc(O)ccc23)O1]

Coriandrum sativum L. is a culinary and medicinal herb of Apiaceae family commonly known as coriander. All parts of the plant are edible, but the fresh leaves/aerial parts and dried seeds are most commonly used traditionally in cooking. The prominent secondary metabolites in coriander include phenolic acids, volatile oils, flavonoids and terpenoids (Tang, E. L. H. et al., BMC Complementary and Alternative Medicine, 2013, 13, 347).

Curcuma amada Roxb (Mango ginger) is a plant of Zingiberaceae family. It is native to India and grows in wild in many states across India. The rhizome of this plant is a unique spice having morphological resemblance with ginger but imparts a raw mango flavor. The main use of Curcuma amada rhizome is in the manufacture of pickles and culinary preparations. Ayurveda and Unani medicinal systems have given lot of importance to Curcuma amada as an appetizer, alexiteric, antipyretic, aphrodisiac, diuretic, emollient, expectorant and laxative and to cure biliousness, itching, skin diseases, bronchitis, asthma, cough and inflammation from injuries. The major secondary metabolites found in rhizome include phenolic acids, volatile oils, curcuminoids and terpenoids (Policegoudra, R. S. et al., Journal of Biosciences, 2011, 36, 739-748). The inventors isolated eight compounds from 90% aqueous ethanol extract of Curcuma amada and the structures of these compounds were elucidated based on their physical and spectroscopic data (NMR, mass etc). The names of these compounds are Zerumin A (1), (E)-15,16-bisnorlabda-8(17), 11-diene-13-one (2), (E)-Labda-8(17), 12-diene-15,16-anhydride (3), (E)-14-hydroxy-15-norlabda-8(17), 12-dien-16-oic acid (4), Zerumin B (5), Coronarin D ethyl ether C-15 epimer (6), Furanodienone (7) and Zederone (8) and their chemical structures are shown below. The 90% aqueous ethanol extract of Curcuma amada was standardized to these eight compounds by HPLC method of analysis and the data was presented in table-2.

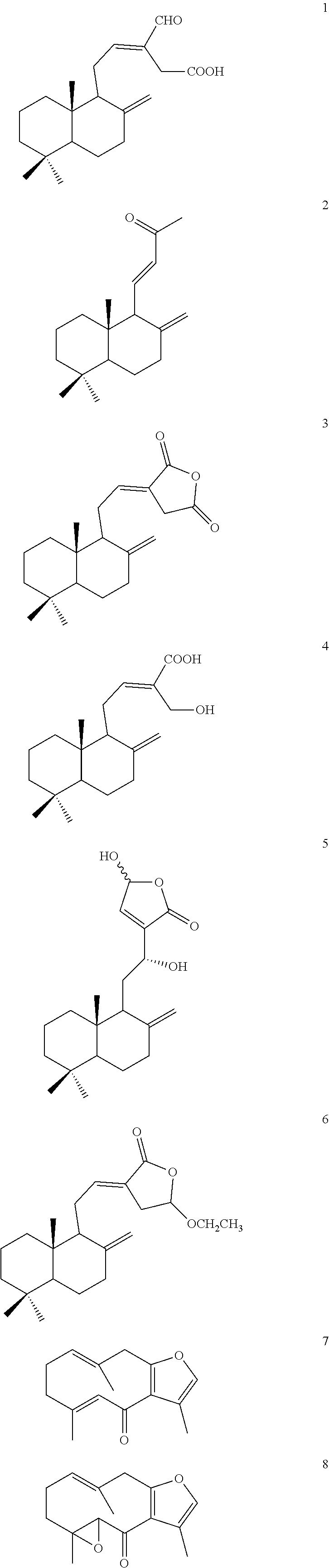

The sources of the raw material used in the current invention are summarized below:—
1. Murraya koenigii collected from Agiripalli village in Krishna district of Andhra Pradesh.
2. Coriandrum sativum Collected from Kunchenapalli village in Guntur district of Andhra Pradesh.
3. Curcuma amada collected from Peraiah gudem village in Bhadradri Kothagudem district of Telangana state.

The dried Murraya koenigii leaves were pulverized and the herb powder was extracted separately with various solvents such as 50% aqueous ethanol, ethanol, water, 50% aqueous acetone, 50% aqueous methanol and ethyl acetate to obtain 50% aqueous ethanol extract (M.K-1), ethanol extract (M.K-2), water extract (M.K-3), 50% aqueous acetone extract (M.K-4), 50% aqueous methanol extract (M.K-5) and ethyl acetate extract (M.K-6) respectively. The said extracts of Murraya koenigii were standardized to Mahanine by analytical HPLC method and the results were summarized in Table 1. Similarly, the dried Coriandrum sativum aerial parts were pulverized and the powder was extracted with various solvents such as 50% aqueous ethanol, ethanol and water to obtain 50% aqueous ethanol extract (C.S-1), ethanol extract (C.S-2) and water extract (C.S-3) respectively. In a similar manner, dried rhizomes of Curcuma amada were pulverized and the powder was extracted with various solvents such as 90% aqueous ethanol, ethanol, 50% aqueous ethanol, water, acetone, methanol and ethyl acetate to obtain 90% aqueous ethanol extract (C.A-1), ethanol extract (C.A-2), 50% aqueous ethanol extract (C.A-3), water extract (C.A-4), acetone extract (C.A-5), methanol extract (C.A-6) and ethyl acetate extract (C.A-7) respectively. The extracts of dried aerial parts of Murraya koenigii and dried leaves of Coriandrum sativum were also prepared using similar procedure.

The extracts of Murraya koenigii, Coriandrum sativum and Curcuma amada were evaluated for their ability to improve SOX-9 expression in cell based assay. Briefly, the SW1353 human chondrosarcoma cells in culture were challenged with IL-1β. Then, the cells were treated with different concentrations of test samples and incubated for 3 hours in a CO₂ incubator. After treatment, cells were evaluated for SOX-9 expression on flow cytometer (BD FACSVerse) using Alexafluor® 647 Mouse anti-human SOX-9 antibody (0.1 μl/well from the stock; BD Pharmingen Cat #565493). Percentage of recovery of experimental damage to the SOX-9 was calculated.

The extracts of Murraya koenigii, Coriandrum sativum and Curcuma amada were evaluated for their ability to inhibit MMP-13 in vitro cellular models. The MMP-13 inhibition was evaluated using SW1353 human chondrosarcoma cells. Briefly, SW1353 cells (10000 cells/well) were seeded in a 96-well plate overnight. Next day, cells were pre-treated with different concentrations of test samples for 2 hours (kept at 37° C. in a CO₂ incubator) and cells with 0.2% DMSO served as vehicle control. After incubation, cells were induced with IL-1β (0.1 ng/ml final concentration) for 24 hours except vehicle control. The cells treated only with IL-1β served as induction control. The culture medium was centrifuged at 250×g and supernatants were tested for MMP-13 quantitation using ELISA Kit (R&D Systems, Cat #DY511). Percentage inhibition of MMP-13 was calculated. The results indicated that the extracts were potent in inhibiting MMP-13.

The LTB₄ inhibitory potential of the extracts was evaluated using polymorphonuclear neutrophils (PMNs) isolated from human peripheral blood. Briefly, PMNs (50000 cells/well in a 96-well plate) were pre-treated with different concentrations of test samples for 2 hours (kept at 37° C. in a CO₂ incubator) and cells treated with 0.2% DMSO served as vehicle control. After incubation, cells were induced with Calcium Ionophore A23187 (10 μM final concentration) for 10 minutes except vehicle control. The cells treated only with A23187 served as induction control. Plate was centrifuged at 250×g and supernatants were tested for LTB₄ quantitation using ELISA Kit (R&D Systems, Cat #SKGE006B). Percentage inhibition of LTB4 was calculated.

Similarly, the PGE₂ inhibition showed by the extracts was evaluated using peripheral blood mononuclear cells (PBMCs) isolated from human peripheral blood. Briefly, PBMCs (0.1×10⁶ cells/well in a 96-well plate) were pre-treated with different concentrations of test samples for 2 hours (kept at 37° C. in a CO₂ incubator) and cells with 0.2% DMSO served as vehicle control. After incubation, cells were induced with Lipopolysaccharide (LPS) (10 ng/ml final concentration) for 4 hours except vehicle control. The cells treated only with LPS served as induction control. Plate was centrifuged at 250×g and supernatants were tested for PGE₂ quantitation using ELISA Kit (Cayman Chemicals, Cat #514010). Percentage inhibition of PGE2 was calculated.

The data from the above in vitro efficacy studies indicated that the extracts of Murraya koenigii, Coriandrum sativum and Curcuma amada have the potential in recovering SOX-9 expression; inhibition of MMP-13, LTB4 and PGE2 in cellular models.

The individual extract(s) or their fraction(s) were then evaluated to check their potential to exhibit synergism when combined. For this purpose, the compositions C-1 to C-55 were prepared by combining different extracts of Murraya koenigii either with Coriandrum sativum extract or Curcuma amada extract in 3:1, 2:1, 1:1, 1:2 and 1:3 ratios. These compositions (compositions 1-55) were then tested in comparison with the corresponding individual extracts for their efficacy in recovering of SOX 9 activity, inhibition of MMP-13, inhibition of PGE2 production and inhibition of LTB4 activities.

The compositions (compositions 1-55) exhibited better efficacy in improving SOX-9 expression in vitro cellular models in comparison with their corresponding individual ingredients. For example, Murraya koenigii 50% aqueous ethanol extract (M.K-1) at 0.75 μg/mL concentration and Coriandrum sativum 50% aqueous ethanol extract (C.S-1) at 0.25 μg/mL concentration showed 34.34% and 8.00% recovery of SOX-9 respectively. The composition-1 containing Murraya koenigii 50% aqueous ethanol extract (M.K-1) and Coriandrum sativum 50% aqueous ethanol extract (C.S-1) in the ratio of 3:1 at 1 μg/mL showed 60.29% recovery of SOX 9, which is significantly better than the additive effect 42.34% (34.34%+8.00%) calculated from the SOX-9 recovery showed by the corresponding individual ingredients. The compositions-2 to 4 (C-2 to C-4) containing these two extracts (M.K-1 and C.S-1) at ratios 2:1, 1:1 and 1:2 respectively also exhibited synergistic SOX9 recovery, when compared to the recoveries exhibited by each of their corresponding individual ingredient concentrations as summarized in Table 3. Similarly, Murraya koenigii 50% aqueous ethanol extract (M.K-1) at 0.75 μg/mL concentration and Curcuma amada 90% aqueous ethanol extract (C.A-1) at 0.25 μg/mL concentration showed 20.60% and 0.03% recovery of SOX-9 respectively. The composition-6 containing Murraya koenigii 50% aqueous ethanol extract (M.K-1) and Curcuma amada 90% aqueous ethanol extract (C.A-1) in the ratio of 3:1 at 1 μg/mL showed 33.88% recovery of SOX 9, which is significantly better than the additive effect 20.63% (20.60%+0.03%) calculated from the SOX-9 recoveries showed by the corresponding individual ingredients. The compositions-7 to 10 (C-7 to C-10) containing these two extracts (M.K-1 and C.A-1) at ratios 2:1, 1:1, 1:2 and 1:3 respectively also exhibited synergism when compared to the SOX-9 recovery shown by each of their corresponding individual ingredient concentrations as summarized in Table 4. The other randomly selected compositions among C11 to C55 containing various extracts of Murraya koenigii in combination either with extracts of Coriandrum sativum or extracts of Curcuma amada also showed synergistic SOX-9 recovery as summarized in Tables 5-7.

The compositions (compositions 1-55) were tested for their efficacy to inhibit MMP-13 in vitro cellular models in comparison with the corresponding individual ingredients. Thus, Murraya koenigii 50% aqueous ethanol extract (M.K-1) at 3.75 g/mL and Coriandrum sativum 50% aqueous ethanol extract (C.S-1) at 1.25 μg/mL concentration showed 9.60% and 6.20% inhibition of MMP-13 respectively. The composition-1 containing Murraya koenigii 50% aqueous ethanol extract (M.K-1) and Coriandrum sativum 50% aqueous ethanol extract (C.S-1) in the ratio of 3:1 at 5 μg/mL showed 23.57% inhibition of MMP-13, which is significantly better than the additive effect 15.80% (9.60%+6.20%) calculated from the inhibition of MMP-13 showed by the corresponding individual ingredients. The other compositions-2 to 5 (C-2 to C-5) containing these two extracts (M.K-1 and C.S-1) at ratios 2:1, 1:1, 1:2 and 1:3 respectively also exhibited synergism when compared to the inhibition of MMP-13 shown by each of their corresponding individual ingredient concentrations as summarized in Table 8. Similarly, Murraya koenigii 50% aqueous ethanol extract (M.K-1) at 0.75 μg/mL and Curcuma amada 90% aqueous ethanol extract (C.A-1) at 0.25 pig/mL concentration showed 6.51% and 3.02% inhibition of MMP-13 respectively. The composition-6 containing Murraya koenigii 50% aqueous ethanol extract (M.K-1) and Curcuma amada 90% aqueous ethanol extract (C.A-1) in the ratio of 3:1 at 1 μg/mL showed 48.36% inhibition of MMP-13, which is significantly higher than the additive effect 9.53% (6.51%+3.02%) calculated from the MMP-13 inhibitory effects shown by the corresponding individual ingredients. The other compositions-7 to 10 (C-7 to C-10) containing these two extracts (M.K-1 and C.A-1) at ratios 2:1, 1:1, 1:2 and 1:3 respectively also exhibited synergism when compared to the MMP-13 inhibition shown by each of their corresponding individual ingredient concentrations as summarized in Table 9. The other randomly selected compositions among C11 to C55 containing various extracts of Murraya koenigii in combination either with extracts of Coriandrum sativum or extracts of Curcuma amada also showed synergistic MMP-13 inhibition (Tables 8-11).

The compositions (compositions 1-55) were also tested for their PGE2 inhibition potential using in vitro cellular models in comparison with the corresponding individual ingredients. For example, Murraya koenigii 50% aqueous ethanol extract (M.K-1) at 7.5 μg/mL and Curcuma amada 90% aqueous ethanol extract (C.A-1) at 2.5 μg/mL concentration showed 16.80% and 21.51% inhibition of PGE2 respectively. The composition-6 containing Murraya koenigii 50% aqueous ethanol extract (M.K-1) and Curcuma amada 90% aqueous ethanol extract (C.A-1) in the ratio of 3:1 at 10 μg/mL showed 47.63% inhibition of PGE2, which is significantly better than the additive effect 38.31% (16.80%+21.51%) calculated from the PGE2 inhibitions shown by the corresponding individual ingredients. The composition-7 to composition-10 containing these two extracts (M.K-1 and C.A-1) at ratios 2:1, 1:1, 1:2 and 1:3 respectively also exhibited synergism when compared to the inhibition of PGE2 shown by each of their corresponding individual ingredient concentrations as summarized in Table 12.

The compositions (compositions 1-55) were further tested for their efficacy to inhibit LTB4 production using in vitro cellular models in comparison with the corresponding individual ingredients. For example, Murraya koenigii 50% aqueous ethanol extract (M.K-1) at 7.5 μg/mL and Coriandrum sativum 50% aqueous ethanol extract (C.S-1) at 2.5 μg/mL concentration showed 52.95% and 1.46% inhibition of LTB4 respectively. The composition-1 containing Murraya koenigii 50% aqueous ethanol extract (M.K-1) and Coriandrum sativum 50% aqueous ethanol extract (C.S-1) in the ratio of 3:1 at 10 μg/mL showed 69.3% inhibition of LTB4, which is significantly better than the additive effect 54.41% (52.95%+1.46%) calculated from the LTB4 inhibitions shown by the corresponding individual ingredients. The composition-3 and composition-5 containing these two extracts (M.K-1 and C.S-1) at ratios 1:1 and 1:3 respectively also exhibited synergism when compared to the inhibition of LTB4 shown by each of their corresponding individual ingredient concentrations as summarized in Table 13. A few other randomly selected compositions among C6 to C55 containing various extracts of Murraya koenigii in combination either with extracts of Coriandrum sativum or extracts of Curcuma amada also showed synergistic LTB4 inhibition (Table 14).

Hence, these compositions (composition 1-55) unexpectedly showed better efficacy to improve SOX-9 recovery/expression, MMP-13 inhibition, PGE2 inhibition and LTB4 inhibition when compared to their corresponding individual ingredients suggesting that these individual extract(s) and fraction(s) have the tendency to show synergism when combined together.

Formulations: The present invention also provides synergistic herbal compositions comprising combination of first ingredient selected from the extract(s), fraction(s), phytochemical(s) and mixtures thereof derived from Murraya koenigii and second ingredient selected from the extract(s), fraction(s), phytochemical(s) and mixtures thereof derived from Curcuma amada or Coriandrum sativum; and optionally containing at least one component selected from pharmaceutically or nutraceutically or dietically acceptable excipients, carriers and diluents.

The compositions comprising combination of first ingredient selected from the extract(s), fraction(s), phytochemical(s) and mixtures thereof derived from Murraya koenigii and second ingredient selected from the extract(s), fraction(s), phytochemical(s) and mixtures thereof derived from Curcuma amada or Coriandrum sativum; and optionally containing at least one component selected from pharmaceutically or nutraceutically or dietically acceptable excipients, carriers and diluents; for improving cartilage growth/repair/regeneration, improving joint mobility, reducing cartilage breakdown, reducing joint pain, reducing stiffness, reducing inflammation and/or treating/alleviating symptoms associated with osteoarthritis; wherein the pharmaceutically or nutraceutically or dietically acceptable excipients, carriers and diluents are selected from monosaccharide's such as glucose, dextrose, fructose, galactose etc.; Disaccharides such as but not limited to sucrose, maltose, lactose, lactulose, trehalose cellobiose, chitobiose etc.; Polycarbohydrates such as Starch and modified starch such as Sodium starch glycolate, pre-gelatinized starch, soluble starch, and other modified starches; Dextrins that are produced by hydrolysis of starch or glycogen such as yellow dextrin, white dextrin, Maltodextrin etc.; Polyhydric alcohols or sugar alcohols such as but not limited to Sorbitol, mannitol, inositol, xylitol, isomalt etc.; cellulose based derivatives such as but not limited to microcrystalline cellulose, hydroxy propyl methyl cellulose, hydroxy ethyl cellulose etc.; silicates such as but not limited to neusilin, veegum, Talc, colloidal silicon dioxide etc.; metallic stearates such as but not limited to calcium stearate, magnesium stearate, zinc Stearate etc.; Organic acids such as citric acid, tartaric acid, malic acid, succinic acid, lactic acid, L-ascorbic acid etc.; Fatty acid esters and esters of poly sorbate, natural gums such as but not limited to acacia, carrageenan, Guar gum, Xanthan gum etc.; vitamin B group, nicotinamide, calcium pantothenate, amino acids, proteins such as but not limited to casein, gelatin, pectin, agar; organic metal salts such as but not limited to sodium chloride, calcium chloride, dicalcium phosphate, zinc sulphate, zinc chloride etc.; Natural pigments, flavors, Class I & Class II preservatives and aqueous, alcoholic, hydro-alcoholic, organic solutions of above listed ingredients alone or in combination.

Efficacy of composition-56 and composition-57 for reducing pain and improving joint architecture in MIA Induced osteoarthritis model: The efficacy shown by the compositions in cellular in vitro models was further evaluated in vivo in Monosodium Iodoacetate (MIA) induced osteoarthritis (OA) model in Sprague Dawley Rats. In this model, osteoarthritis-like pathology and joint pain were induced in the rats by a single intra-articular administration of MIA into the right hind knee joint.

Improvement in weight-bearing capacity: The MIA-induced group (G2) rats showed significant reduction of the body-weight bearing capacity (expressed as percentage) on the right hind limb as compared to the normal-saline group (G1). The reduced body-weight bearing capacity in G2 was sustained through the end of the study. In contrast, Composition-56 (G3) and Composition-57 (G4) supplemented rats showed significantly improved weight-bearing capacities at day 28, when compared with MIA induced rats (G2) (FIG. I). These data indicate that Composition-56 and Composition-57 supplementation helped to reduce knee pain on their affected right hind knee joints.

Improvement in paw withdrawal latency to thermal stimulus: The latency of the right hind paw withdrawal in MIA group (G2) was significantly decreased when compared with the vehicle control (G1). The paw withdrawal latency of the right hind limb exhibited by the treatment groups supplemented with composition-56 (G3), and composition-57 (G4) was significantly increased when compared with the MIA-induced animals (G2) (FIG. II).

Improvement in paw withdrawal threshold: MIA-induced animals (G2) showed substantially decreased pressure threshold in comparison with the vehicle control animals (G1) on day 5, and it sustained till the end of the study (FIG. III). On day 27, the animals in the treatment groups G3 and G4 supplemented with composition-56 and composition-57 respectively showed significant improvements in pressure threshold in comparison with that of the MIA-induced animals (G2) (FIG. III).

Together, these observations suggest that Composition-56 and Composition-57 supplementation significantly reduced sensitivity to pain perception and resulting in significantly improved osteoarthritis-related clinical symptoms in the experimentally induced OA in rats.

Improvement in joint architecture: The experimental rats in control and the supplemented groups were sacrificed 28 days post-injection of MIA. The left hind limbs were disarticulated, and knees were excised, processed, embedded in paraffin wax. The paraffin-embedded tissue sections were mounted on slides and were processed and stained following the standard procedure. The stained tissue sections were examined for histopathological changes under an optical microscope. The presence of chondrocytes (indicated by arrow) and ECM staining are evident in the vehicle control (G1). The tissue samples in G2 group had shown damaged articular cartilage, characterized by complete loss of cellular detail and some loss of extracellular matrix components as indicated by light red stain (FIG. IV). The tissue sections of animals supplemented with Composition-56 (G3) and Composition-57 (G4) exhibited a large number of chondrocytes and adequate ECM stain, which indicate that these inventive compositions could protect the cartilage through preventing the MIA induced damage and help maintain healthy joint (FIG. IV).

The forgoing demonstrates that synergistic herbal compositions comprising combination of first ingredient selected from the extract(s), fraction(s), phytochemical(s) and mixtures thereof derived from Murraya koenigii and second ingredient selected from the extract(s), fraction(s), phytochemical(s) and mixtures thereof derived from Coriandrum sativum or Curcuma amada unexpectedly showed better efficacy in SOX-9 recovery, MMP-13 inhibition, PGE2 inhibition and LTB4 inhibition. Hence, the said compositions can be useful for obtaining at least one health benefit selected from improving cartilage growth/repair/regeneration, improving joint mobility, reducing cartilage breakdown, reducing joint pain, reducing stiffness, reducing inflammation and/or treating/alleviating symptoms associated with osteoarthritis.

Therefore, in an important embodiment, the present invention provides synergistic herbal compositions comprising combination of a first ingredient selected from the extract(s), fraction(s), phytochemical(s) and mixtures thereof derived from Murraya koenigii and a second ingredient selected from the extract(s), fraction(s), phytochemical(s) and mixtures thereof derived from Coriandrum sativum or Curcuma amada for obtaining at least one health benefit selected from improving cartilage growth/repair/regeneration, improving joint mobility, reducing cartilage breakdown, reducing joint pain, reducing stiffness, reducing inflammation and/or treating/alleviating symptoms associated with osteoarthritis.

In another embodiment, the present invention provides synergistic herbal compositions comprising combination of a first ingredient selected from the extract(s), fraction(s), phytochemical(s) and mixtures thereof derived from Murraya koenigii and a second ingredient selected from the extract(s), fraction(s), phytochemical(s) and mixtures thereof derived from Coriandrum sativum or Curcuma amada for obtaining at least one health benefit selected from improving cartilage growth/repair/regeneration, improving joint mobility, reducing cartilage breakdown, reducing joint pain, reducing stiffness, reducing inflammation and/or treating/alleviating symptoms associated with osteoarthritis; wherein the concentration of the first ingredient in the composition varies in the range of 10%-90% by weight and the concentration of the second ingredient varies in the range of 90%-10% by weight.

In one specific aspect, the invention provides synergistic composition comprising combination of first ingredient selected from the extract(s), fraction(s), phytochemical(s) and mixtures thereof derived from Murraya koenigii and second ingredient selected from the extract(s), fraction(s), phytochemical(s) and mixtures thereof derived from Coriandrum sativum wherein the concentration of the extracts or fractions, phytochemicals of Murraya koenigii in the composition varies in the range of 10%-90% by weight and the concentration of the extracts or fractions, phytochemicals of Coriandrum sativum varies in the range of 90%-10% by weight.

In another specific aspect, the invention provides synergistic composition comprising combination of first ingredient selected from the extract(s), fraction(s), phytochemical(s) and mixtures thereof derived from Murraya koenigii and second ingredient selected from the extract(s), fraction(s), phytochemical(s) and mixtures thereof derived from Curcuma amada wherein the concentration of the extracts or fractions, phytochemicals of Murraya koenigii in the composition varies in the range of 10%-90% by weight and the concentration of the extracts or fractions, phytochemicals of Curcuma amada varies in the range of 90%-10% by weight.

In other exemplary embodiment, the present invention provides synergistic herbal compositions comprising combination of first ingredient selected from the extract(s), fraction(s), phytochemical(s) and mixtures thereof derived from Murraya koenigii and second ingredient selected from the extract(s), fraction(s), phytochemical(s) and mixtures thereof derived from Coriandrum sativum or Curcuma amada and optionally comprises at least one component selected from pharmaceutically or nutraceutically or dietically acceptable excipients, carriers and diluents for obtaining at least one health benefit selected from improving cartilage growth/repair/regeneration, improving joint mobility, reducing cartilage breakdown, reducing joint pain, reducing stiffness, reducing inflammation and/or treating/alleviating symptoms associated with osteoarthritis.

In another embodiment, the present invention provides synergistic herbal compositions comprising combination of first ingredient selected from the extract(s), fraction(s), phytochemical(s) and mixtures thereof derived from Murraya koenigii and second ingredient selected from the extract(s), fraction(s), phytochemical(s) and mixtures thereof derived from Coriandrum sativum or Curcuma amada and optionally comprises at least one component selected from pharmaceutically or nutraceutically or dietically acceptable excipients, carriers and diluents; for obtaining at least one health benefit selected from improving cartilage growth/repair/regeneration, improving joint mobility, reducing cartilage breakdown, reducing joint pain, reducing stiffness, reducing inflammation and/or treating/alleviating symptoms associated with osteoarthritis; wherein the pharmaceutically or nutraceutically or dietically acceptable excipients, carriers and diluents are selected from monosaccharide's such as glucose, dextrose, fructose, galactose etc.; Disaccharides such as but not limited to sucrose, maltose, lactose, lactulose, trehalose cellobiose, chitobiose etc.; Polycarbohydrates such as Starch and modified starch such as Sodium starch glycolate, pre gelatinized starch, soluble starch, and other modified starches; Dextrins that are produced by hydrolysis of starch or glycogen such as yellow dextrin, white dextrin, Maltodextrin etc.; Polyhydric alcohols or sugar alcohols such as but not limited to Sorbitol, mannitol, inositol, xylitol, isomalt etc.; cellulose based derivatives such as but not limited to microcrystalline cellulose, hydroxy propyl methyl cellulose, hydroxy ethyl cellulose etc.; silicates such as but not limited to neusilin, veegum, Talc, colloidal silicon dioxide etc.; metallic stearates such as but not limited to calcium stearate, magnesium stearate, zinc Stearate etc.; Organic acids such as citric acid, tartaric acid, malic acid, succinic acid, lactic acid, L-ascorbic acid etc.; Fatty acid esters and esters of poly sorbate, natural gums such as but not limited to acacia, carrageenan, Guar gum, Xanthan gum etc.; vitamin B group, nicotinamide, calcium pantothenate, amino acids, proteins such as but not limited to casein, gelatin, pectin, agar; organic metal salts such as but not limited to sodium chloride, calcium chloride, dicalcium phosphate, zinc Sulphate, zinc chloride etc.; Natural pigments, flavors, Class I & Class II preservatives and aqueous, alcoholic, hydro-alcoholic, organic solutions of above listed ingredients alone or in combination.

In another embodiment, the invention provides synergistic herbal compositions comprising combination of a first ingredient selected from the extract(s), fraction(s), phytochemical(s) and mixtures thereof derived from Murraya koenigii and a second ingredient selected from the extract(s), fraction(s), phytochemical(s) and mixtures thereof derived from Coriandrunm satirum or Curcuma amada, wherein the extract or fraction is obtained from at least one plant part selected from the group comprising leaves, stems, tender stems, tender twigs, aerial parts, whole fruit, fruit peel rind, seeds, flower heads, root, bark, hardwood, rhizome or whole plant or mixtures thereof.

In another embodiment, the invention provides synergistic herbal compositions comprising combination of a first ingredient selected from the extract(s), fraction(s), phytochemical(s) and mixtures thereof derived from Murraya koenigii and a second ingredient selected from the extract(s), fraction(s), phytochemical(s) and mixtures thereof derived from Coriandrum sativum or Curcuma amada, wherein the extract or fraction is produced using at least one solvent selected from the group comprising C1-C5 alcohols like ethanol, methanol, n-propanol, isopropyl alcohol; ketones like acetone, methyl isobutyl ketone, chlorinated solvents like methylene dichloride and chloroform, water and mixtures thereof; C1-C7 hydrocarbons such as hexane; esters like ethyl acetate and the like and mixtures thereof.

In the other embodiment, the present invention provides synergistic herbal compositions comprising combination of a first ingredient selected from the extract(s), fraction(s), phytochemical(s) and mixtures thereof derived from Murraya koenigii and a second ingredient selected from the extract(s), fraction(s), phytochemical(s) and mixtures thereof derived from Coriandrum sativum or Curcuma amada, wherein compositions are standardized to at least one phytochemical reference marker compound or biological active marker in the extract or fraction; wherein phytochemical marker compound or group of phytochemical compounds is in the concentration range of 0.1% to 99% by weight of the extract.

In another embodiment, the present invention provides synergistic herbal compositions comprising combination of first ingredient selected from the extract(s), fraction(s), phytochemical(s) and mixtures thereof derived from Murraya koenigii and second ingredient selected from the extract(s), fraction(s), phytochemical(s) and mixtures thereof derived from Coriandrum sativum or Curcuma amada; wherein the compositions are standardized to at least one alkaloid compound of Murraya koenigii, which include but not limited to mahanine, mahanimbine, isomahanine, mahanibidine, koenimbine, murrayanol; wherein Mahanine is in the concentration range of 0.1% to 10% by weight of the composition

In another embodiment, the present invention provides synergistic herbal compositions comprising combination of first ingredient selected from the extract(s), fraction(s), phytochemical(s) and mixtures thereof derived from Murraya koenigii and second ingredient selected from the extract(s), fraction(s), phytochemical(s) and mixtures thereof derived from Coriandrum sativum or Curcuma amada; wherein the composition(s) are formulated into a dosage form selected from dry powder form, liquid form, beverage, food product, dietary supplement or any suitable form such as tablet, a capsule, a soft chewable or gummy bear.

In another embodiment of the invention, the composition(s) as disclosed above can be formulated into nutritional/dietary supplements that can be contemplated/made into the dosage form of healthy foods, or food for specified health uses such as solid food like chocolate or nutritional bars, semisolid food like cream, jam, or gel or beverage such as refreshing beverage, lactic acid bacteria beverage, drop, candy, chewing gum, gummy candy, yoghurt, ice cream, pudding, soft adzuki bean jelly, jelly, cookie, tea, soft drink, juice, milk, coffee, cereal, snack bar and the like.

In a further embodiment, the present invention provides methods of obtaining at least one health benefit selected from improving cartilage growth/repair/regeneration, improving joint mobility, reducing cartilage breakdown, reducing joint pain, reducing stiffness, reducing inflammation and/or treating/alleviating symptoms associated with osteoarthritis in a human, wherein the method comprises supplementing the said human with an effective dose of a composition comprising combination of a first ingredient selected from the extract(s), fraction(s), phytochemical(s) and mixtures thereof derived from Murraya koenigii and a second ingredient selected from the extract(s), fraction(s), phytochemical(s) and mixtures thereof derived from Coriandrum sativum or Curcuma amada; and optionally containing at least one component selected from pharmaceutically or nutraceutically or dietically acceptable excipients, carriers and diluents.

The effective dose of the compositions according to the invention can be in the form of oral, topical or parenteral compositions. The oral dosage forms may be further formulated as solid or liquid dosage forms. The solid dosage forms include tablets, capsules, pallets, powders, granules and can be formulated as controlled release dosage forms using controlled release polymers or polymer-based coatings by the techniques including nanotechnology, microencapsulation, colloidal carrier systems and other drug delivery systems for obtaining the desired therapeutic benefit.

In another embodiment, the present invention provides methods of ameliorating at least one biomarker selected from SOX-9, MMP-13, PGE2 and LTB4 for obtaining health benefit selected from improving cartilage growth/repair/regeneration, improving joint mobility, reducing cartilage breakdown, reducing joint pain, reducing stiffness, reducing inflammation and/or treating/alleviating symptoms associated with osteoarthritis in humans, wherein the method comprises supplementing the said human with an effective dose of a composition comprising combination of a first ingredient selected from the extract(s), fraction(s), phytochemical(s) and mixtures thereof derived from Murraya koenigii and a second ingredient selected from the extract(s), fraction(s), phytochemical(s) and mixtures thereof derived from Coriandrum sativum or Curcuma amada; and optionally containing at least one component selected from pharmaceutically or nutraceutically or dietically acceptable excipients, carriers and diluents.

In another embodiment, the present invention provides use of synergistic herbal compositions comprising combination of a first ingredient selected from the extract(s), fraction(s), phytochemical(s) and mixtures thereof derived from Murraya koenigii and a second ingredient selected from the extract(s), fraction(s), phytochemical(s) and mixtures thereof derived from Coriandrum sativum or Curcuma amada; and optionally containing at least one component selected from pharmaceutically or nutraceutically or dietically acceptable excipients, carriers and diluents for obtaining at least one health benefit selected from improving cartilage growth/repair/regeneration, improving joint mobility, reducing cartilage breakdown, reducing joint pain, reducing stiffness, reducing inflammation and/or treating/alleviating symptoms associated with osteoarthritis.

Those of ordinary skilled in the art will appreciate that changes could be made to the embodiments described above without departing from the broad inventive concept thereof. It is understood, therefore, that this invention is not limited to the particular embodiments or examples disclosed herein, but is intended to cover modifications within the objectives and scope of the present invention as defined in the specification. The presented examples illustrate the invention, but they should not be considered to limit the scope of the invention in any way.

### Example 1: Preparation of Murraya koenigii Extract

(a) 50% aqueous ethanol extract: Murraya koenigii leaf dried raw material (100 g) was pulverized and the herb powder was extracted with 50% aqueous ethanol (700 mL) at rt for 1 h. The extract was filtered and the spent raw material was re-extracted twice with 50% aqueous ethanol (2×500 mL) under similar conditions. The combined extract was filtered and concentrated under vacuum to obtain a residue of 50% aqueous ethanol extract as dry powder (M.K-1; 15.9 g).
(b) Ethanol and water extracts: The ethanol extract (M.K-2; 9.0 g) and water extract (M.K-3; 16.6 g) were obtained from 100 g raw material by adopting similar procedure using ethanol and water as extraction solvents respectively.
(c) 50% aqueous acetone extract: Murraya koenigii leaf dried raw material (100 g) was pulverized and the herb powder was extracted with 50% aqueous acetone (700 mL) at rt for 1 h. The extract was filtered and the spent raw material was re-extracted twice with 50% aqueous acetone (2×500 mL) under similar conditions. The combined extracts were filtered and concentrated under vacuum to obtain a residue of 50% aqueous acetone extract as a greenish brown color dry powder (M.K-4; 14.2 g).
(d) 50% aqueous methanol extract: Murraya koenigii leaf dried raw material (100 g) was pulverized and the herb powder was extracted with 50% aqueous ethanol (700 mL) at rt for 1 h. The extract was filtered and the spent raw material was re-extracted twice with 50% aqueous methanol (2×500 mL) under similar conditions. The combined extracts were filtered and concentrated under vacuum to obtain a residue of 50% aqueous methanol extract as a greenish brown color dry powder (M.K-5; 14.1 g).
(e) Ethyl acetate extract: Murraya koenigii leaf dried raw material (100 g) was pulverized and the herb powder was extracted with ethyl acetate (700 mL) at rt for 1 h. The extract was filtered and the spent raw material was re-extracted twice with 50% aqueous ethanol (2×500 mL) under similar conditions. The combined extracts were filtered and concentrated under vacuum to obtain a residue of ethyl acetate extract as a greenish brown color thick paste (M.K-6; 3.3 g).

Standardization: The above Murraya koenigii leaf extracts were standardized to Mahanine, a carbazole alkaloid, by analytical HPLC method and the results are summarized below in Table 1.

TABLE 1
Details of Murraya koenigii extracts

| | | Solvent for | Weight of the | Mahanine |
|---|---|---|---|---|
| S. No. | Extract code | extraction | product | (HPLC) |
| 1 | M.K-1 | 50% aqueous | 15.9 g | 1.4% |
| | | ethanol | | |
| 2 | M.K-2 | ethanol | 9.0 g | 5.05% |
| 3 | M.K-3 | water | 16.6 g | 0.05% |
| 4 | M.K-4 | 50% aqueous | 14.2 g | 3.05% |
| | | acetone | | |
| 5 | M.K-5 | 50% aqueous | 14.1 g | 1.52% |
| | | methanol | | |
| 6 | M.K-6 | Ethyl acetate | 3.3 g | 12.54% |

### Example 2: Preparation of Coriandrum sativum Extract

(a) 50% aqueous ethanol extract: Coriandrum sativum aerial parts dried raw material (100 g) was pulverized and the powder was extracted with 50% aqueous ethanol (700 mL) at rt for 1 h. The extract was filtered and the spent raw material was re-extracted twice with 50% aqueous ethanol (2×500 mL) under similar conditions. The combined extract was filtered and concentrated under vacuum to obtain a residue of 50% aqueous ethanol extract as dark brown color powder (C.S-1; 12.4 g).
(b) Ethanol and water extracts: The ethanol extract (C.S-2; 4.0 g) and water extract (C.S-3; 16.7 g) were obtained from 100 g raw material by adopting similar extraction procedure as above using ethanol and water as extraction solvents respectively.

### Example 3: Preparation of Curcuma amada Extract

(a) 90% aqueous ethanol extract: Curcuma amada rhizomes dried raw material (100 g) was pulverized and the powder was extracted with 90% aqueous ethanol (700 mL) at rt for 1 h. The extract was filtered and the spent raw material was re-extracted twice with 90% aqueous ethanol (2×500 mL) under similar conditions. The combined extract was filtered and concentrated under vacuum to obtain a residue of 90% aqueous ethanol extract as dark brown color powder (C.A-1; 11.1 g).
(b) Ethanol, 50% aqueous ethanol and water extracts: The ethanol extract (C.A-2; 7.1 g), 50% aqueous ethanol extract (C.A-3; 19.2 g) and water extract (C.A-4; 11.6 g) were obtained from 100 g raw material by adopting similar extract procedure as above using ethanol, 50% aqueous ethanol and water as extraction solvents respectively.
(c) Acetone extract: Curcuma amada rhizomes dried raw material (100 g) was pulverized and the powder was extracted with acetone (700 mL) at rt for 1 h. The extract was filtered and the spent raw material was re-extracted twice with acetone (2×500 mL) under similar conditions. The combined extracts were filtered and concentrated under vacuum to obtain a residue of acetone extract as brown color thick paste (C.A-5; 3.9 g).
(d) Methanol extract: Curcuma amada rhizomes dried raw material (100 g) was pulverized and the powder was extracted with methanol (700 mL) at rt for 1 h. The extract was filtered and the spent raw material was re-extracted twice with methanol (2×500 mL) under similar conditions. The combined extracts were filtered and concentrated under vacuum to obtain a residue of methanol extract as brown color thick paste (C.A-6; 4.3 g).
(e) Ethyl acetate extract: Curcuma amada rhizomes dried raw material (100 g) was pulverized and the powder was extracted with ethyl acetate (700 mL) at rt for 1 h. The extract was filtered and the spent raw material was re-extracted twice with ethyl acetate (2×500 mL) under similar conditions. The combined extracts were filtered and concentrated under vacuum to obtain a residue of ethyl acetate extract as a brown color powder (C.A-7; 4.0 g).

TABLE 2
HPLC data of Curcuma amada extract

| | | HPLC of 90% |
|---|---|---|
| | | ethanol extract |
| S. No. | Marker Compound (Phytochemical) name | (C.A-1) |
| 1 | Zerumin A | 0.78% |
| 2 | (E)-15,16-bisnorlabda-8(17), ll-diene-13-one | 0.20% |
| 3 | (E)-Labda-8(17),12-diene-15,16-anhydride | 0.21% |
| 4 | (E)-14-hydroxy-15-norlabda-8(17), 12-dien- | 0.57% |
| | 16-oic acid | |
| 5 | Zerumin B | 0.05% |
| 6 | Coronarin D ethyl ether C-15 epimer | 0.56% |
| 7 | Furanodienone | 0.53% |
| 8 | Zederone | 0.12% |

### Example 4: Preparation of Various Compositions Containing Murraya koenigii Leaf 50% Aqueous Ethanol Extract (M.K-1) and Coriandrum sativum Aerial Parts 50% Aqueous Ethanol Extract (C.S-1)

Composition-1 (C-1): The composition-1 was prepared by combining Murraya koenigii 50% aqueous ethanol extract (M.K-1) and Coriandrum sativum 50% aqueous ethanol extract (C.S-1) in the ratio of 3:1.

Composition-2 (C-2): The composition-2 was prepared by combining Murraya koenigii 50% aqueous ethanol extract (M.K-1) and Coriandrum sativum 50% aqueous ethanol extract (C.S-1) in the ratio of 2:1.

Composition-3 (C-3): The composition-3 was prepared by combining Murraya koenigii 50% aqueous ethanol extract (M.K-1) and Coriandrum sativum 50% aqueous ethanol extract (C.S-1) in the ratio of 1:1.

Composition-4 (C-4): The composition-4 was prepared by combining Murraya koenigii 50% aqueous ethanol extract (M.K-1) and Coriandrum sativum 50% aqueous ethanol extract (C.S-1) in the ratio of 1:2.

Composition-5 (C-5): The composition-5 was prepared by combining Murraya koenigii 50% aqueous ethanol extract (M.K-1) and Coriandrum sativum 50% aqueous ethanol extract (C.S-1) in the ratio of 1:3.

### Example 5: Preparation of Various Compositions of Murraya koenigii Leaf 50′% Aqueous Ethanol Extract (M.K-1) and Curcuma amada Rhizome 90% Aqueous Ethanol Extract (C.A-1)

Composition-6 (C-6): The composition-6 was prepared by combining Murraya koenigii 50% aqueous ethanol extract (M.K-1) and Curcuma amada 90% aqueous ethanol extract (C.A-1) in the ratio of 3:1.

Composition-7 (C-7): The composition-7 was prepared by combining Murraya koenigii 50% aqueous ethanol extract (M.K-1) and Curcuma amada 90% aqueous ethanol extract (C.A-1) in the ratio of 2:1.

Composition-8 (C-8): The composition-8 was prepared by combining Murraya koenigii 50% aqueous ethanol extract (M.K-1) and Curcuma amada 90% aqueous ethanol extract (C.A-1) in the ratio of 1:1.

Composition-9 (C-9): The composition-9 was prepared by combining Murraya koenigii 50% aqueous ethanol extract (M.K-1) and Curcuma amada 90% aqueous ethanol extract (C.A-1) in the ratio of 1:2.

Composition-10 (C-10): The composition-10 was prepared by combining Murraya koenigii 50% aqueous ethanol extract (M.K-1) and Curcuma amada 90% aqueous ethanol extract (C.A-1) in the ratio of 1:3.

### Example 6: Preparation of Various Compositions of Murraya koenigii Leaf 50% Aqueous Methanol Extract (M.K-5) and Coriandrum sativum Aerial Parts 50% Aqueous Ethanol Extract (C.S-2)

Composition-11 (C-11): The composition-11 was prepared by combining Murraya koenigii 50% aqueous methanol extract (M.K-5) and Coriandrum sativum 50% aqueous ethanol extract (C.S-2) in the ratio of 3:1.

Composition-12 (C-12): The composition-12 was prepared by combining Murraya koenigii 50% aqueous methanol extract (M.K-5) and Coriandrum sativum 50% aqueous ethanol extract (C.S-2) in the ratio of 2:1.

Composition-13 (C-13): The composition-13 was prepared by combining Murraya koenigii 50% aqueous methanol extract (M.K-5) and Coriandrum sativum 50% aqueous ethanol extract (C.S-2) in the ratio of 1:1.

Composition-14 (C-14): The composition-14 was prepared by combining Murraya koenigii 50% aqueous methanol extract (M.K-5) and Coriandrum sativum 50% aqueous ethanol extract (C.S-2) in the ratio of 1:2.

Composition-15 (C-15): The composition-15 was prepared by combining Murraya koenigii 50% aqueous methanol extract (M.K-5) and Coriandrum sativum 50% aqueous ethanol extract (C.S-2) in the ratio of 1:3.

### Example 7: Preparation of Various Compositions of Murraya koenigii Leaf Water Extract (M.K-3) and Curcuma amada Rhizome Ethanol Extract (C.A-2)

Composition-16 (C-16): The composition-16 was prepared by combining Murraya koenigii water extract (M.K-3) and Curcuma amada ethanol extract (C.A-2) in the ratio of 3:1.

Composition-17 (C-17): The composition-17 was prepared by combining Murraya koenigii water extract (M.K-3) and Curcuma amada ethanol extract (C.A-2) in the ratio of 2:1.

Composition-18 (C-18): The composition-18 was prepared by combining Murraya koenigii water extract (M.K-3) and Curcuma amada ethanol extract (C.A-2) in the ratio of 1:1.

Composition-19 (C-19): The composition-19 was prepared by combining Murraya koenigii water extract (M.K-3) and Curcuma amada ethanol extract (C.A-2) in the ratio of 1:2.

Composition-20 (C-20): The composition-20 was prepared by combining Murraya koenigii water extract (M.K-3) and Curcuma amada ethanol extract (C.A-2) in the ratio of 1:3.

### Example 8: Preparation of Various Compositions of Murraya koenigii Leaf 50% Aqueous Acetone Extract (M.K-4) and Curcuma amada Rhizome Water Extract (C.A-4)

Composition-21 (C-21): The composition-21 was prepared by combining Murraya koenigii 50% aqueous acetone extract (M.K-4) and Curcuma amada water extract (C.A-4) in the ratio of 3:1.

Composition-22 (C-22): The composition-22 was prepared by combining Murraya koenigii 50% aqueous acetone extract (M.K-4) and Curcuma amada water extract (C.A-4) in the ratio of 2:1.

Composition-23 (C-23): The composition-23 was prepared by combining Murraya koenigii 50% aqueous acetone extract (M.K-4) and Curcuma amada water extract (C.A-4) in the ratio of 1:1.

Composition-24 (C-24): The composition-24 was prepared by combining Murraya koenigii 50% aqueous acetone extract (M.K-4) and Curcuma amada water extract (C.A-4) in the ratio of 1:2.

Composition-25 (C-25): The composition-25 was prepared by combining Murraya koenigii 50% aqueous acetone extract (M.K-4) and Curcuma amada water extract (C.A-4) in the ratio of 1:3.

### Example 9: Preparation of Various Compositions of Murraya koenigii Leaf 50% Aqueous Methanol Extract (M.K-5) and Curcuma amada Rhizome Ethyl Acetate Extract (C.A-7)

Composition-26 (C-26): The composition-26 was prepared by combining Murraya koenigii 50% aqueous methanol extract (M.K-5) and Curcuma amada ethyl acetate extract (C.A-7) in the ratio of 3:1.

Composition-27 (C-27): The composition-27 was prepared by combining Murraya koenigii 50% aqueous methanol extract (M.K-5) and Curcuma amada ethyl acetate extract (C.A-7) in the ratio of 2:1.

Composition-28 (C-28): The composition-28 was prepared by combining Murraya koenigii 50% aqueous methanol extract (M.K-5) and Curcuma amada ethyl acetate extract (C.A-7) in the ratio of 1:1.

Composition-29 (C-29): The composition-29 was prepared by combining Murraya koenigii 50% aqueous methanol extract (M.K-5) and Curcuma amada ethyl acetate extract (C.A-7) in the ratio of 1:2.

Composition-30 (C-30): The composition-30 was prepared by combining Murraya koenigii 50% aqueous methanol extract (M.K-5) and Curcuma amada ethyl acetate extract (C.A-7) in the ratio of 1:3.

### Example 10: Preparation of Various Compositions of Murraya koenigii Leaf Ethyl Acetate Extract (M.K-6) and Curcuma amada Rhizome Acetone Extract (C.A-5)

Composition-31 (C-31): The composition-31 was prepared by combining Murraya koenigii ethyl acetate extract (M.K-6) and Curcuma amada acetone extract (C.A-5) in the ratio of 3:1.

Composition-32 (C-32): The composition-32 was prepared by combining Murraya koenigii ethyl acetate extract (M.K-6) and Curcuma amada acetone extract (C.A-5) in the ratio of 2:1. Composition-33 (C-33): The composition-33 was prepared by combining Murraya koenigii ethyl acetate extract (M.K-6) and Curcuma amada acetone extract (C.A-5) in the ratio of 1:1. Composition-34 (C-34): The composition-34 was prepared by combining Murraya koenigii ethyl acetate extract (M.K-6) and Curcuma amada acetone extract (C.A-5) in the ratio of 1:2. Composition-35 (C-35): The composition-35 was prepared by combining Murraya koenigii ethyl acetate extract (M.K-6) and Curcuma amada acetone extract (C.A-5) in the ratio of 3:1.

### Example 11: Preparation of Various Compositions of Murraya koenigii Leaf Ethanol Extract (M.K-2) and Curcuma amada Rhizome Methanol Extract (C.A-6)

Composition-36 (C-36): The composition-36 was prepared by combining Murraya koenigii ethanol extract (M.K-2) and Curcuma amada methanol extract (C.A-6) in the ratio of 3:1.

Composition-37 (C-37): The composition-37 was prepared by combining Murraya koenigii ethanol extract (M.K-2) and Curcuma amada methanol extract (C.A-6) in the ratio of 2:1.

Composition-38 (C-38): The composition-38 was prepared by combining Murraya koenigii ethanol extract (M.K-2) and Curcuma amada methanol extract (C.A-6) in the ratio of 1:1.

Composition-39 (C-39): The composition-39 was prepared by combining Murraya koenigii ethanol extract (M.K-2) and Curcuma amada methanol extract (C.A-6) in the ratio of 1:2.

Composition-40 (C-40): The composition-40 was prepared by combining Murraya koenigii ethanol extract (M.K-2) and Curcuma amada methanol extract (C.A-6) in the ratio of 1:3.

### Example 12: Preparation of Various Compositions of Murraya koenigii Leaf 50% Aqueous Methanol Extract (M.K-5) and Curcuma amada Rhizome 50% Aqueous Ethanol Extract (C.A-3)

Composition-41 (C-41): The composition-41 was prepared by combining Murraya koenigii 50% aqueous methanol extract (M.K-5) and Curcuma amada 50% aqueous ethanol extract (C.A-3) in the ratio of 3:1.

Composition-42 (C-42): The composition-42 was prepared by combining Murraya koenigii 50% aqueous methanol extract (M.K-5) and Curcuma amada 50% aqueous ethanol extract (C.A-3) in the ratio of 2:1.

Composition-43 (C-43): The composition-43 was prepared by combining Murraya koenigii 50% aqueous methanol extract (M.K-5) and Curcuma amada 50% aqueous ethanol extract (C.A-3) in the ratio of 1:1.

Composition-44 (C-44): The composition-44 was prepared by combining Murraya koenigii 50% aqueous methanol extract (M.K-5) and Curcuma amada 50% aqueous ethanol extract (C.A-3) in the ratio of 1:2.

Composition-45 (C-45): The composition-45 was prepared by combining Murraya koenigii 50% aqueous methanol extract (M.K-5) and Curcuma amada 50% aqueous ethanol extract (C.A-3) in the ratio of 1:3.

### Example 13: Preparation of Various Compositions of Murraya koenigii Leaf 50% Aqueous Ethanol Extract (M.K-1) and Coriandrum sativum Aerial Parts Water Extract (C.S-3)

Composition-46 (C-46): The composition-46 was prepared by combining Murraya koenigii 50% aq ethanol extract (M.K-1) and Coriandrum sativum water extract (C.S-3) in the ratio of 3:1.

Composition-47 (C-47): The composition-47 was prepared by combining Murraya koenigii 50% aq ethanol extract (M.K-1) and Coriandrum sativum water extract (C.S-3) in the ratio of 2:1.

Composition-48 (C-48): The composition-48 was prepared by combining Murraya koenigii 50% aq ethanol extract (M.K-1) and Coriandrum sativum water extract (C.S-3) in the ratio of 1:1.

Composition-49 (C-49): The composition-49 was prepared by combining Murraya koenigii 50% aq ethanol extract (M.K-1) and Coriandrum sativum water extract (C.S-3) in the ratio of 1:2.

Composition-50 (C-50): The composition-50 was prepared by combining Murraya koenigii 50% aq ethanol extract (M.K-1) and Coriandrum sativum water extract (C.S-3) in the ratio of 1:3.

### Example 14: Preparation of Various Compositions of Murraya koenigii Leaf 50% Aqueous Methanol Extract (M.K-5) and Curcuma amada Rhizome 90% Aq Ethanol Extract (C.A-1)

Composition-51 (C-51): The composition-51 was prepared by combining Murraya koenigii leaf 50% aqueous methanol extract (M.K-5) and Curcuma amada rhizome 90% aq ethanol extract (C.A-1) in the ratio of 3:1.

Composition-52 (C-52): The composition-52 was prepared by combining Murraya koenigii leaf 50% aqueous methanol extract (M.K-5) and Curcuma amada rhizome 90% aq ethanol extract (C.A-1) in the ratio of 2:1.

Composition-53 (C-53): The composition-53 was prepared by combining Murraya koenigii leaf 50% aqueous methanol extract (M.K-5) and Curcuma amada rhizome 90% aq ethanol extract (C.A-1) in the ratio of 1:1.

Composition-54 (C-54): The composition-54 was prepared by combining Murraya koenigii leaf 50% aqueous methanol extract (M.K-5) and Curcuma amada rhizome 90% aq ethanol extract (C.A-1) in the ratio of 1:2.

Composition-55 (C-55): The composition-55 was prepared by combining Murraya koenigii leaf 50% aqueous methanol extract (M.K-5) and Curcuma amada rhizome 90% aq ethanol extract (C.A-1) in the ratio of 1:3.

### Example 15: Formulation of the Compositions

Composition-56 (C-56): The composition-56 was prepared by combining 60 g of Murraya koenigii leaf 50% aqueous ethanol extract (M.K-1), 30 g of Coriandrum sativun aerial parts 50% aqueous ethanol extract (C.S-1), 9 g of Maltodextrin and 1 g of syloid in presence of water and dried to give the composition-56.

Composition-57 (C-57): The composition-57 was prepared by combining 60 g of Murraya koenigii 50% aqueous ethanol extract (M.K-1), 20 g of Curcuma amada 90% aqueous ethanol extract (C.A-1), 5 g of Ultrasperse-A, 13 g of Maltodextrin and 2 g of syloid in presence of water and dried to give composition-57.

### Example 16: Assay for SRY-Box 9 (SOX-9) Recovered

In a 96 well plate, forty thousand SW1353 cells were seeded with 150 μL of DMEM supplemented with 10% Fetal Bovine Serum (FBS). All the wells were induced with 10 μL of IL-1β (1 ng/mL) except control (Cells+0.2% DMSO) and incubated them at 37° C. for 1 hour in a CO₂ incubator. Cells treated only with IL-1β served as induction control. Then the wells were treated with 50 μL respective concentrations of test samples and incubated them at 37° C. for 3 hours in a CO₂ incubator. After treatment, cells were transferred into 96 well “V” bottom plate and the plate was centrifuged at 1300 rpm for 5 minutes at 4° C. Supernatant was discarded carefully through aspiration and cells were washed once with 200 μl of 1×PBS and the centrifugation step was repeated again. After discarding the PBS, 200 μL of fix perm solution was added to all the wells and incubated for 20 minutes in the dark. Centrifugation was repeated and the supernatant was discarded. To the cell pellet, 200 μL of fix perm solution was added once again to all the wells and incubated for 15 minutes in the dark. The plate was centrifuged at 1300 rpm for 5 minutes at 4° C. and supernatant was aspirated carefully and 20p L of diluted Alexafluor® 647 Mouse anti-human SOX-9 antibody (0.1 μl/well from the stock; BD Pharmingen Cat #565493) was added to all the wells except unstained (added with 20 μl of perm buffer) and incubated in dark for 30 minutes. Supernatant was discarded after centrifugation at 4° C. and cells were washed with FACS buffer (1×PBS+2% FBS) and repeated with the centrifugation step again. Finally, 300 μL of FACS buffer was added to all the wells and analyzed in a BD FACSVerse flow cytometer. Percentage of recovery of experimental damage to the SOX-9 was calculated using the following formula.

% SOX-9 Recovered=[(Normalized value of % SOX-9 in the test)−(Normalized value of % SOX-9 in induction)]/[(Normalized value of % SOX-9 in the stained)−(Normalized value of % SOX-9 in induction)]×100.

The results are presented in Tables 3-7.

TABLE 3
SOX-9 recovery of the compositions containing Murraya koenigii
(M.K-1) and Coriandrum sativum (C.S-1)

| | | | | | | | % Recovery | |
|---|---|---|---|---|---|---|---|---|
| | | | | | | | of SOX9 | |
| | M.K-1 | | C.S-1 | | | Comp | production | |
| Comp | | % | | % | | Dose | Additive | |
| # | μg/mL | Recovery | μg/mL | Recovery | Ratio | μg/mL | (Calculated) | Observed |
| C-1 | 0.75 | 34.34 | 0.25 | 8.00 | 3:1 | 1 | 42.34 | 60.29 |
| C-2 | 0.67 | 30.67 | 0.33 | 10.56 | 2:1 | 1 | 41.23 | 78.56 |
| C-3 | 0.5 | 22.89 | 0.5 | 16.00 | 1:1 | 1 | 38.89 | 43.08 |
| C-4 | 0.33 | 15.11 | 0.67 | 21.44 | 1:2 | 1 | 36.55 | 75.98 |

TABLE 4
SOX-9 recovery of the compositions containing Murraya koenigii
(M.K-1) and Curcuma amada (C.A-1)

| | | | | | | | % Recovery | |
|---|---|---|---|---|---|---|---|---|
| | | | | | | | of SOX9 | |
| | M.K-1 | | C.A-1 | | | Comp | production | |
| Comp | | % | | % | | Dose | Additive | |
| # | μg/mL | Recovery | μg/mL | Recovery | Ratio | μg/mL | (Calculated) | Observed |
| C-6 | 0.75 | 20.60 | 0.25 | 0.03 | 3:1 | 1 | 20.63 | 33.88 |
| C-7 | 0.67 | 18.40 | 0.33 | 0.04 | 2:1 | 1 | 18.44 | 24.23 |
| C-8 | 0.5 | 13.73 | 0.5 | 0.07 | 1:1 | 1 | 13.80 | 22.06 |
| C-9 | 0.33 | 9.06 | 0.67 | 0.09 | 1:2 | 1 | 9.15 | 17.88 |
| C-10 | 0.25 | 6.87 | 0.75 | 0.10 | 1:3 | 1 | 6.96 | 17.51 |

TABLE 5
SOX-9 recovery of the compositions containing Murraya koenigii
(M.K-5) and Coriandrum sativum (C.S-2)

| | | | | | | | % Recovery | |
|---|---|---|---|---|---|---|---|---|
| | | | | | | | of SOX9 | |
| | M.K-5 | | C.S-2 | | | Comp | production | |
| Comp | | % | | % | | Dose | Additive | |
| # | μg/mL | Recovery | μg/mL | Recovery | Ratio | μg/mL | (Calculated) | Observed |
| C-11 | 3.75 | 24.71 | 1.25 | 10.44 | 3:1 | 5 | 35.16 | 55.21 |
| C-13 | 2.5 | 16.52 | 2.5 | 20.72 | 1:1 | 5 | 37.24 | 70.62 |
| C-15 | 1.25 | 8.33 | 3.75 | 31.00 | 1:3 | 5 | 39.33 | 48.63 |

TABLE 6
SOX-9 recovery of the compositions containing Murraya koenigii
(M.K-3, M.K-4, M.K-5) and Curcuma amada (CA-2, C.A-4, C.A-7)

| | | | | | | | % Recovery | |
|---|---|---|---|---|---|---|---|---|
| | | | | | | | of SOX9 | |
| | | | | | | Comp | production | |
| Comp | | % | | % | | Dose | Additive | |
| # | μg/mL | Recovery | μg/mL | Recovery | Ratio | μg/mL | (Calculated) | Observed |
| | M.K-3 | | C.A-2 | | | | | |
| C-18 | 0.5 | 23.56 | 0.5 | 9.33 | 1:1 | 1 | 32.89 | 47.99 |
| | M.K-4 | | C.A-4 | | | | | |
| C-23 | 0.5 | 20.45 | 0.5 | 20.07 | 1:1 | 1 | 40.52 | 49.34 |
| | M.K-5 | | C.A-7 | | | | | |
| C-26 | 1.87 | 59.61 | 0.63 | 5.41 | 3:1 | 2.5 | 65.02 | 74.24 |
| C-27 | 1.67 | 53.23 | 0.83 | 7.12 | 2:1 | 2.5 | 60.36 | 73.61 |
| C-28 | 1.25 | 39.85 | 1.25 | 10.73 | 1:1 | 2.5 | 50.58 | 63.78 |

TABLE 7
SOX-9 recovery of the compositions containing Murraya koenigii
(M.K-6, M.K-2, MK-5) and Curcuma amada (C.A-5, C.A-6, C.A-3)

| | | | | | | | % Recovery | |
|---|---|---|---|---|---|---|---|---|
| | | | | | | | of SOX-9 | |
| | | | | | | Comp | production | |
| Comp | | % | | % | | Dose | Additive | |
| # | μg/mL | Recovery | μg/mL | Recovery | Ratio | μg/mL | (Calculated) | Observed |
| | M.K-6 | | C.A-5 | | | | | |
| C-31 | 0.75 | 69.74 | 0.25 | 7.40 | 3:1 | 1 | 77.14 | 83.38 |
| C-32 | 0.67 | 62.30 | 0.33 | 9.76 | 2:1 | 1 | 72.07 | 87.7 |
| C-33 | 0.5 | 46.50 | 0.5 | 14.80 | 1:1 | 1 | 61.29 | 77.6 |
| | M.K-2 | | C.A-6 | | | | | |
| C-36 | 0.75 | 70.92 | 0.25 | 4.87 | 3:1 | 1 | 75.79 | 81.77 |
| C-37 | 0.67 | 63.36 | 0.33 | 6.43 | 2:1 | 1 | 69.78 | 72.56 |
| C-38 | 0.5 | 47.28 | 0.5 | 9.74 | 1:1 | 1 | 57.02 | 68.47 |
| | M.K-5 | | C.A-3 | | | | | |
| C-41 | 0.75 | 27.81 | 0.25 | 3.22 | 3:1 | 1 | 31.03 | 48.83 |
| C-43 | 0.67 | 24.84 | 0.33 | 4.25 | 2:1 | 1 | 29.09 | 39.41 |
| C-43 | 0.5 | 18.54 | 0.5 | 6.44 | 1:1 | 1 | 24.98 | 34.69 |

### Example 17: Assay for Matrix Metalloproteinase-13 (MMP-13) Inhibition

In a 96 well plate, ten thousand SW1353 cells were seeded with 200 μL of DMEM medium supplemented with 10% FBS and incubated overnight at 37° C. in a CO₂ incubator. Next day, the plate was washed twice with basal DMEM medium (without FBS). Cells were pretreated with different concentrations of test samples. Cells with 0.2% DMSO served as a vehicle control. The plate was incubated in a CO₂ incubator at 37° C. for 2 hrs. After due incubation, all the wells were induced with IL-1β at a final concentration of 0.1 ng/mL except vehicle control and incubated at 37° C. in a CO₂ incubator for 24 hrs. Cells treated only with IL-1β served as induction control. Plate was centrifuged at 270×g for 5 minutes and cell-free culture supernatants were collected. Quantitation of MMP-13 was performed using ELISA (R&D Duoset, Human total MMP-13 Cat #DY511) according to the manufacturer's instructions. Absorbance was measured at 450 nm in a Spectramax2e plate reader. Inhibition of MMP-13 was calculated using the following formula.

% Inhibition of MMP-13=[(Normalized Conc. of MMP13 in Induction)−(Normalized Conc. of MMP13 in Test sample)]/(Normalized Conc. of MMP13 in Induction)×100

The results are presented in Tables 8-11.

TABLE 8
MMP-13 inhibition of the compositions containing Murraya koenigii
(M.K-1) and Coriandrum sativum (C.S-1 & C.S-2).

| | | | | | | | % Inhibition | |
|---|---|---|---|---|---|---|---|---|
| | | | | | | | of MMP13 | |
| | | | | | | Comp | production | |
| Comp | | % | | % | | Dose | Additive | |
| # | μg/mL | Inhibition | μg/mL | Inhibition | Ratio | μg/mL | (Calculated) | Observed |
| | M.K-1 | | C.S-1 | | | | | |
| C-1 | 3.75 | 9.60 | 1.25 | 6.20 | 3:1 | 5 | 15.80 | 23.57 |
| C-2 | 3.33 | 8.52 | 1.67 | 8.28 | 2:1 | 5 | 16.80 | 32.66 |
| C-3 | 2.5 | 6.40 | 2.5 | 12.40 | 1:1 | 5 | 18.80 | 30.69 |
| C-4 | 0.83 | 4.79 | 1.67 | 7.30 | 1:2 | 2.5 | 12.10 | 28.43 |
| C-5 | 0.63 | 3.64 | 1.87 | 8.18 | 1:3 | 2.5 | 11.81 | 31.3 |
| | M.K-5 | | C.S-2 | | | | | |
| C-11 | 0.75 | 8.30 | 0.25 | 0.64 | 3:1 | 1 | 8.93 | 16.49 |
| C-13 | 2.5 | 6.49 | 2.5 | 13.65 | 1:1 | 5 | 20.14 | 32.27 |
| C-14 | 0.25 | 2.77 | 0.75 | 1.91 | 1:3 | 1 | 4.67 | 20.21 |

TABLE 9
MMP-13 inhibition of the compositions containing Murraya koenigii
(M.K-1) and Curcuma amada (C.A-1).

| | | | | | | | % Inhibition | |
|---|---|---|---|---|---|---|---|---|
| | | | | | | | of MMP13 | |
| | M.K-1 | | C.A-1 | | | Comp | production | |
| Comp | | % | | % | | Dose | Additive | |
| # | μg/mL | Inhibition | μg/mL | Inhibition | Ratio | μg/mL | (Calculated) | Observed |
| C-6 | 0.75 | 6.51 | 0.25 | 3.02 | 3:1 | 1 | 9.53 | 48.36 |
| C-7 | 0.67 | 5.82 | 0.33 | 3.99 | 2:1 | 1 | 9.80 | 47.4 |
| C-8 | 0.5 | 4.34 | 0.5 | 6.04 | 1:1 | 1 | 10.38 | 21.16 |
| C-9 | 0.33 | 2.86 | 0.67 | 8.09 | 1:2 | 1 | 10.96 | 34.38 |
| C-10 | 0.25 | 2.17 | 0.75 | 9.06 | 1:3 | 1 | 11.23 | 26.36 |

TABLE 10
MMP-13 inhibition of the compositions containing Murraya koenigii
(MK-3, M.K-4, M.K-5) and Curcuma amada (C.A-2, C.A-4, C.A-7).

| | | | | | | | % Inhibition | |
|---|---|---|---|---|---|---|---|---|
| | | | | | | | of MMP13 | |
| | | | | | | Comp | production | |
| Comp | | % | | % | | Dose | Additive | |
| # | μg/mL | Inhibition | μg/mL | Inhibition | Ratio | μg/mL | (Calculated) | Observed |
| | M.K-3 | | C.A-2 | | | | | |
| C-18 | 0.5 | 0.24 | 0.5 | 23.66 | 1:1 | 1 | 23.90 | 32.66 |
| | M.K-4 | | C.A-4 | | | | | |
| C-23 | 5 | 16.37 | 5 | 24.12 | 1:1 | 10 | 40.49 | 54.78 |
| | M.K-5 | | C.A-7 | | | | | |
| C-26 | 1.87 | 6.76 | 0.63 | 8.80 | 3:1 | 2.5 | 15.56 | 30.68 |
| C-27 | 6.67 | 19.56 | 3.33 | 11.20 | 2:1 | 10 | 30.76 | 45.52 |
| C-28 | 1.25 | 4.52 | 1.25 | 17.46 | 1:1 | 2.5 | 21.98 | 35.23 |

TABLE 11
MMP-13 inhibition of the compositions containing Murraya koenigii
(M.K-6, M.K-2, M.K-5) and Curcuma amada (C.A-5, C.A-6, CA-3).

| | | | | | | | % Inhibition | |
|---|---|---|---|---|---|---|---|---|
| | | | | | | | of MMP13 | |
| | | | | | | Comp | production | |
| Comp | | % | | % | | Dose | Additive | |
| # | μg/mL | Inhibition | μg/mL | Inhibition | Ratio | μg/mL | (Calculated) | Observed |
| | M.K-6 | | C.A-5 | | | | | |
| C-31 | 3.75 | 21.92 | 1.25 | 7.21 | 3:1 | 5 | 29.12 | 40.87 |
| C-32 | 3.33 | 19.46 | 1.67 | 9.63 | 2:1 | 5 | 29.09 | 44.92 |
| C-33 | 2.5 | 14.61 | 2.5 | 14.42 | 1:1 | 5 | 29.03 | 44.63 |
| | M.K-2 | | C.A-6 | | | | | |
| C-36 | 3.75 | 14.52 | 1.25 | 3.19 | 3:1 | 5 | 17.71 | 39.32 |
| C-37 | 3.33 | 12.89 | 1.67 | 4.26 | 2:1 | 5 | 17.16 | 27.34 |
| C-38 | 2.5 | 9.68 | 2.5 | 6.38 | 1:1 | 5 | 16.06 | 32.85 |
| | M.K-5 | | C.A-3 | | | | | |
| C-41 | 0.75 | 00 | 0.25 | 4.34 | 3:1 | 1 | 4.34 | 26.68 |
| C-42 | 6.67 | 13.90 | 3.33 | 7.35 | 2:1 | 10 | 21.25 | 34.17 |

### Example 18: Assay for Prostaglandin E₂ (PGE₂) Inhibition

Human blood was collected from healthy volunteers from peripheral vein with a syringe containing EDTA at final concentration of 2 mM. Plasma was separated by centrifugation at 1000 rpm for 10 minutes and the residual blood was diluted with RPMI medium supplemented with 10% FBS and 2 mM EDTA in a ratio of 1:3. Thirty milliliters of blood was carefully layered onto the 15 mL of ficoll/Lymphoprep in a 50 mL falcon tube in dark and tubes were centrifuged at 350×g for 30 minutes at acceleration of 9 without using brake. Buffy coat (interface between medium and ficoll) containing peripheral blood mononuclear cells (PBMC) was collected carefully in 25 mL of cold 1× phosphate buffered saline (PBS) and centrifuged at 1200 rpm for 10 minutes. Residual RBCs found in PBMCs pellet was removed by treating with ACK lysis buffer (Gibco Cat #A10492-01) and washed with fresh 1×PBS. PBMC were seeded in a 96-well plate with a density of 0.1×10⁶ cells/well and treated with different concentrations of test samples. Cells with 0.2% DMSO served as a vehicle control. The plate was incubated in a CO₂ incubator at 37° C. for 2 hrs. Finally, cells were induced with LPS (10 ng/mL final concentration) for 4 hours except for vehicle control by keeping the plate at 37° C. in a CO₂ incubator. Cells treated only with LPS served as induction control. Plate was centrifuged at 1200 rpm for 5 minutes and 120 μL cell-free supernatants were collected. Quantitation of PGE₂ was performed using ELISA kit (Cayman Chemicals Cat #514010) according to the manufacturer's instructions. Absorbance was measured at 412 nm in a kinetic mode for 30 minutes in a Spectramax2e plate reader. Inhibition of PGE₂ was calculated using the following formula.

% Inhibition of PGE₂=[(Normalized Conc. of PGE₂ in Induction)−(Normalized Conc. of PGE₂ in Test sample)]/(Normalized Conc. of PGE₂ in Induction)×100

Note: Excess PBMC were cryopreserved in liquid nitrogen using cell freezing medium at a density of 5×10⁶ cells per tube. When the cryopreserved PBMC were thawed for the experiment, they were rested overnight in RPMI+10% FBS medium in a CO₂ incubator and proceeded for treatment the next morning. The results are presented in Table-12.

TABLE 12
PGE2 inhibition of the compositions containing Murraya koenigii
(M.K-1, M.K-5) and Curcuma amada (C.A-1)

| | | | | | | | % Inhibition | |
|---|---|---|---|---|---|---|---|---|
| | | | | | | | of PGE2 | |
| | | | | | | Comp | production | |
| Comp | | % | | % | | Dose | Additive | |
| # | μg/mL | inhibition | μg/mL | inhibition | Ratio | μg/mL | (Calculated) | Observed |
| | M.K-1 | | C.A-1 | | | | | |
| C-6 | 7.5 | 16.80 | 2.5 | 21.51 | 3:1 | 10 | 38.31 | 47.63 |
| C-7 | 6.67 | 14.94 | 3.33 | 28.65 | 2:1 | 10 | 43.59 | 52.69 |
| C-8 | 5 | 11.20 | 5 | 43.02 | 1:1 | 10 | 54.22 | 69.87 |
| C-9 | 3.33 | 7.46 | 6.67 | 57.39 | 1:2 | 10 | 64.85 | 80.34 |
| C-10 | 2.5 | 5.60 | 7.5 | 64.53 | 1:3 | 10 | 70.13 | 95.31 |
| | M.K-5 | | C.A-1 | | | | | |
| C-51 | 1.87 | 24.68 | 0.63 | 13.74 | 3:1 | 2.5 | 38.42 | 37.88 |
| C-55 | 0.63 | 8.32 | 1.87 | 40.78 | 1:3 | 2.5 | 49.10 | 44.95 |

### Example 19: Assay for Leukotriene B4 (LTB4) Inhibition

Human blood was collected from healthy volunteers from peripheral vein with a syringe containing EDTA at final concentration of 2 mM. Plasma was separated by centrifugation at 1000 rpm for 10 minutes and the residual blood was diluted with RPMI medium supplemented with 10% FBS and 2 mM EDTA in a ratio of 1:3. Thirty milliliters of blood was carefully layered onto the 15 mL of ficoll/Lymphoprep in a 50 mL falcon tube in dark and tubes were centrifuged at 350×g for 30 minutes at acceleration of 9 without using brake. After removing peripheral blood mononuclear cells (PBMC) and ficoll/Lymphoprep, settled RBC layer containing granulocytes was treated with ACK lysis buffer (Gibco Cat #A10492-01) to lyse the RBC completely. After centrifugation at 1200 rpm for 10 minutes, the resulting cell pellet of polymorphonuclear leukocytes (PMNs) was resuspended with RPMI+1% newborn calf serum (NBCS). These cells were seeded in 96-well plate at a density of 50,000 cells/well and treated with different concentrations of test samples. Cells with 0.2% DMSO served as a vehicle control. The plate was incubated in a CO₂ incubator at 37° C. for 2 hrs. Finally, cells treated with test samples were induced with A23187 (10 μM final concentration) for 10 minutes by keeping the plate at 37° C. in a CO₂ incubator. Cells treated only with A23187 served as induction control. Plate was centrifuged at 1200 rpm for 5 minutes and 120p L of cell-free supernatants were collected. Quantitation of LTB₄ was performed using LTB4 ELISA kit (R&D Systems, Cat #SKGE006B) according to manufacturer's instructions. Absorbance was measured at 450 nm with correction wavelength of 570 nm in a Spectramax2e plate reader. Inhibition of LTB4 was calculated using the following formula.

% Inhibition of LTB₄=[(Normalized Conc. of LTB₄ in Induction)−(Normalized Conc. of LTB₄ in Test sample)]/(Normalized Conc. of LTB₄ in Induction)×100

The results are presented in Tables 13-14.

TABLE 13
LTB4 inhibition of the compositions containing Murraya koenigii
(M.K-1) and Coriandrum sativum (C.S-1)

| | | | | | | | % Inhibition | |
|---|---|---|---|---|---|---|---|---|
| | | | | | | | of LTB4 | |
| | M.K-1 | | C.S-1 | | | Comp | production | |
| Comp | | % | | % | | Dose | Additive | |
| # | μg/mL | inhibition | μg/mL | inhibition | Ratio | μg/mL | (Calculated) | Observed |
| C-1 | 7.5 | 52.95 | 2.5 | 1.46 | 3:1 | 10 | 54.41 | 69.3 |
| C-3 | 5 | 35.30 | 5 | 2.92 | 1:1 | 10 | 38.22 | 41.12 |
| C-5 | 2.5 | 17.65 | 7.5 | 4.37 | 1:3 | 10 | 22.02 | 36.75 |

TABLE 14
LTB4 inhibition of the compositions containing Murraya koenigii
(M.K-1, M.K-5) and Curcuma amada (C.A-1)

| | | | | | | | % Inhibition | |
|---|---|---|---|---|---|---|---|---|
| | | | | | | | of LTB4 | |
| | | | | | | Comp | production | |
| Comp | | % | | % | | Dose | Additive | |
| # | μg/mL | inhibition | μg/mL | inhibition | Ratio | μg/mL | (Calculated) | Observed |
| | M.K-1 | | C.A-1 | | | | | |
| C-6 | 7.5 | 52.95 | 2.5 | 20.34 | 3:1 | 10 | 73.29 | 86.01 |
| C-7 | 6.67 | 47.09 | 3.33 | 27.09 | 2:1 | 10 | 74.18 | 84.26 |
| C-8 | 5 | 35.30 | 5 | 40.67 | 1:1 | 10 | 75.97 | 85.1 |
| C-9 | 3.33 | 23.51 | 6.67 | 54.25 | 1:2 | 10 | 77.76 | 87.51 |
| C-10 | 2.5 | 17.65 | 7-5 | 61.01 | 1:3 | 10 | 78.66 | 89.53 |
| | M.K-5 | | C.A-1 | | | | | |
| C-52 | 6.67 | 15.06 | 3.33 | 27.09 | 2:1 | 10 | 42.15 | 56.3 |
| C-53 | 5 | 11.29 | 5 | 40.67 | 1:1 | 10 | 51.96 | 59.33 |
| C-54 | 3.33 | 7.52 | 6.67 | 54.25 | 1:2 | 10 | 61.77 | 83.95 |

### Example 20: Evaluation of Anti-Osteoarthridc Efficacy of Composition-56 and Composition-57 in Monosodium Iodoacetate (MIA) Induced OA Model in Sprague Dawley Rats

Animals: Preclinical efficacy of Composition-56 and Composition-57 to alleviate osteoarthritic pain and to improve cartilage architecture was evaluated in a monosodium iodoacetate (MIA) induced osteoarthritis (OA) in Sprague-Dawley rats. Female rats of 8-12 weeks of age were housed in standard environmental conditions with a 12-h light/dark cycle at 22±3° C. and 55±15% humidity. All rats received standard rodent diet and water ad libitum. The study was performed in compliance with the guidelines of the Committee for the Purpose of Control and Supervision of Experiments on Animals (CPCSEA), and it was approved by the Institutional Animal Ethics Committee (IAEC).

Experimental study: Monosodium Iodoacetate (MIA) injection (1 mg/25 μL) was used to induce OA in the rats. The MIA was administered into the intra-articular space of the right hind knee of the anesthetized rats. The rats were randomly selected and divided into the following groups based on the right hind limb weight-bearing potential after three days of MIA injection; each group contained six rats. Group 1 (G1): Vehicle control animals received (intra-articular) sterile normal saline; Group 2 (G2): MIA induced control; Group 3 (G3) and Group 4 (G4): MIA induced animals supplemented with Composition-56 (250 mg/kg) and Composition-57 (250 mg/kg), respectively. The experimental rats received oral administration of only CMC (G1 & G2) or CMC mixed with Composition-56 (G3) or Composition-57 (G4) from day 4 to day 28.

Estimation of percent weight-bearing on the right hind limb: An incapacitance tester (Incapacitance meter, IITC, USA) was used to assess changes in their body weight-bearing tolerance. To assess the hind limb weight-bearing distribution, the rats were placed in a measuring chamber for 5 sec for the determination of weight-bearing force. Weight-bearing measurements were done on day 4 (basal) and day 28 of the experiment. The weight distribution ratio of the rats was estimated using the following formula:

Percent wt. bearing of right hind limb=(wt. on the right hind limb×100)/(wt. On right hind limb+wt. on left hind limb).

Composition-56 (G3) and Composition-57 (G4) treated rats showed significantly improved weight-bearing capacities at day 28 as compared with MIA induced rats (G2) as shown in FIG. I. This data indicate that Composition-56 and Composition-57 supplementation helped to normalize the natural body weight-bearing capacity of the animals on their affected right hind limbs.

Estimation of thermal hyperalgesia: Paw withdrawal latency (s) is an indicator of pain sensitivity to heat stimulus, was measured on Days 5 (basal), and 27 (after treatment) by Hargreave's method using a Plantar Analgesiometer, (UgoBasile, ITALY). Time (s) taken (latency) for right hind paw withdrawal after the application of 30 IR heat stimulus was recorded with plantar test apparatus and compared to that of MIA control. The higher the latency period, the more effective is the test item. The paw withdrawal latency (s) of the right hind limb exhibited by the treatment groups supplemented with composition-56 (G3), and composition-57 (G4) was significantly increased when compared with the MIA-induced animals (G2) as summarized in FIG. II.

Estimation of mechanical allodynia: Paw withdrawal threshold (measured as gram-force, gf), an indicator of pain sensitivity to non-noxious stimuli, was measured on Days 5 (basal) and 27 (after treatment) by Von Frey method using an Electronic Von Frey apparatus (IITC, USA). The pressure (gram force) at which the animals withdrew their right hind paw after the application of mechanical stimulus was recorded. The greater the threshold, the more effective is the test item. The animals in the treatment groups G3 and G4 supplemented with composition-56, and composition-57 respectively showed significant improvements in pressure threshold in comparison with that of the MIA-induced animals (G2) as depicted in FIG. III.

Histopathology: After completing the experimental study, rats in all groups were sacrificed by CO₂ asphyxiation. The right knee joint was removed from all rats. The tissues were fixed in 10% formalin, the fixed tissues were embedded in paraffin, and 5 μm thin tissue sections were prepared using a microtome (Leica Biosystems, Germany). The tissue sections were reacted with Safranin O to stain the ECM components of the cartilage followed by fast green to counter-stain the remaining sites. The stained sections were examined under a light microscope (Carl Zeiss GmbH, Germany). The MIA group rats (G2) exhibited histopathological changes such as inflammatory cells in the articular tissue, synovial space, cartilage erosion, and synovial hyperplasia. In contrast, treatment with composition-56 (G3) or composition-57 (G4) exhibits a large number of chondrocytes, thereby confirming that these compositions suppress the synovial hyperplasia and tissue damage in joints. These histopathological changes confirm that composition-56 and composition-57 attenuate the severity of MIA-induced OA in rats. The results are depicted in FIG. IV.

Statistical analysis: Data are presented as mean±standard error (SE). All data analysis was performed using a Two-way analysis of variance (ANOVA), and the Bonferroni test was performed to identify the significance using GraphPad Prism Software.

1-15. (canceled)

16. An herbal composition comprising a combination of:
a first ingredient selected from the group consisting of extracts of Murraya koenigii; and
a second ingredient selected from the group consisting of:
extracts of Coriandrum sativum;
extracts of Curcuma amada, and
mixtures thereof;
wherein the combination comprises 10% to 90% by weight of the first ingredient and 10% to 90% by weight of the second ingredient, based on the combined weight of the first ingredient and the second ingredient.

17. The herbal composition as claimed in claim 16, wherein the combination comprises 25% to 75% by weight of the first ingredient and 25% to 75% by weight of the second ingredient.

18. The herbal composition as claimed in claim 16, wherein the second ingredient is an extract of Coriandrum sativum.

19. The herbal composition as claimed in claim 16, wherein the second ingredient is an extract of Curcuma amada.

20. The herbal composition as claimed in claim 18, wherein the combination comprises 25% to 75% by weight of the first ingredient and 25% to 75% by weight of the second ingredient.

21. The herbal composition as claimed in claim 20, wherein the combination comprises 33% to 75% by weight of the first ingredient and 25% to 67% by weight of the second ingredient.

22. The herbal composition as claimed in claim 20, wherein the combination comprises 33% to 75% by weight of the first ingredient and 25% to 67% by weight of the second ingredient.

23. The herbal composition as claimed in claim 19, wherein the combination comprises 50% to 75% by weight of the first ingredient and 25% to 50% by weight of the second ingredient.

24. The herbal composition as claimed in claim 23, wherein the combination comprises 50% to 75% by weight of the first ingredient and 25% to 50% by weight of the second ingredient.

25. The herbal composition as claimed in claim 16, wherein:
the herbal composition contains at least one alkaloid compound of Murraya koenigii,
the at least one alkaloid compound is:
mahanine; or
a mixture of mahinine and a compound selected from the group consisting of mahanimbine, isomahanine, mahanibidine, koenimbine, murrayanol, and mixtures thereof; and
the herbal composition contains 0.1% to 10% mahanine, based on the weight of the herbal composition.

26. The herbal composition as claimed in claim 16, wherein the herbal composition further comprises at least one excipient, carrier, or diluent.

27. The herbal composition as claimed in claim 16, wherein the herbal composition further comprises an aqueous, alcoholic, hydro-alcoholic, or organic solution of at least one excipient, carrier, or diluent.

28. The herbal composition as claimed in claim 26, wherein the excipient, carrier, or diluent is selected from the group consisting of monosaccharides, disaccharides, polycarbohydrates, dextrins, polyhydric alcohols, sugar alcohols, cellulose derivatives, silicates, stearate salts, organic acids, fatty acid esters, polysorbate esters, natural gums, vitamins, amino acids, proteins, inorganic metal salts, natural pigments, flavors, Class I & Class II preservatives, and combinations thereof.

29. The herbal composition as claimed in claim 26, wherein the excipient, carrier, or diluent is selected from the group consisting of glucose, dextrose, fructose, galactose, sucrose, maltose, lactose, lactulose, trehalose, cellobiose, chitobiose, starch, modified starch, sodium starch glycolate, pregelatinized starch, soluble starch, yellow dextrin, white dextrin, maltodextrin, alginates, sorbitol, mannitol, inositol, xylitol, isomalt, microcrystalline cellulose, hydroxypropyl methyl cellulose, hydroxyethyl cellulose, neusilin, veegum, talc, colloidal silicon dioxide, calcium stearate, magnesium stearate, zinc stearate, citric acid, tartaric acid, malic acid, succinic acid, lactic acid, L-ascorbic acid, fatty acid esters, polysorbate esters, acacia, carrageenan, Guar gum, xanthan gum, vitamin B, nicotinamide, calcium pantothenate, amino acids, casein, gelatin, pectin, agar, sodium chloride, calcium chloride, sodium hydroxide, potassium chloride, potassium hydroxide, dicalcium phosphate, zinc sulphate, zinc chloride, natural pigments, flavors, Class I & Class II preservatives, and combinations thereof.

30. The herbal composition as claimed in claim 16, wherein the first ingredient and the second ingredient are obtained by solvent extraction of at least one plant part selected from the group consisting of leaves, stems, tender stems, tender twigs, aerial parts, whole fruit peel, fruit peel rind, seeds, flower heads, roots, bark, hardwood, rhizomes, a whole plant, and mixtures thereof.

31. The herbal composition as claimed in claim 16, wherein the first ingredient and the second ingredient are obtained by solvent extraction of at least one plant part using a solvent selected from the group consisting of C1-C5 alcohols, ketones, chlorinated solvents, C1-C7 hydrocarbons, esters, water, and mixtures thereof.

32. The herbal composition as claimed in claim 16, wherein the first ingredient and the second ingredient are obtained by solvent extraction of at least one plant part using a solvent selected from the group consisting of ethanol, methanol, n-propanol, isopropyl alcohol, acetone, methyl isobutyl ketone, methylene dichloride, chloroform, hexane, ethyl acetate, water, and mixtures thereof.

33. A dosage form comprising the herbal composition as claimed in claim 16, wherein the dosage form is selected from the group consisting of a dry powder, a liquid, a beverage, a water dispersible formulation, a food product, a dietary supplement, a tablet, a capsule, a soft chewable, and a gummy bear.

34. A dosage form comprising the herbal composition as claimed in claim 16, wherein the dosage form is selected from the group consisting of healthy foods, chocolate bars, nutritional bars, water dispersible beverages, semisolid foods, creams, jams, gels, beverages, lactic acid bacteria-containing beverages, drops, candies, chewing gums, gummy candies, yoghurts, ice creams, puddings, soft adzuki bean jellies, jellies, cookies, teas, soft drinks, juices, milk, coffee, cereals, and snack bars.

35. A method of improving cartilage growth/repair/regeneration, improving joint mobility, reducing cartilage breakdown, reducing joint pain, reducing stiffness, reducing inflammation and/or treating/alleviating symptoms associated with osteoarthritis in humans or animals, wherein the method comprises supplementing the said human or animal with an effective dose of an herbal composition as claimed in claim 16.

36. The method as claimed in claim 13, wherein the herbal composition is provided in a dosage form selected from the group consisting of tablets, capsules, controlled release tablets, and controlled release polymer-coated dosage forms.
